(19) United States
(12) Patent Application Publication
(10) Pub. No.: US 2005/0288288 A1
(43) Pub. Date: Dec. 29, 2005

(54) Methods for preparing P2X7 inhibitors

(75) Inventors: Jason Albert Leonard, Rocky Hill, CT (US); Zheng Jane Li, Quaker Hill, CT (US); Zhengong Bryan Li, East Lyme, CT (US); Frank John Urban, Old Saybrook, CT (US)

Correspondence Address: WARNER-LAMBERT COMPANY, 2800 PLYMOUTH RD, ANN ARBOR, MI, 48105, US

(73) Assignee: PFIZER INC.

(21) Appl. No.: 11/167,786

(22) Filed: Jun. 27, 2005

Related U.S. Application Data
(60) Provisional application No. 60/583,813, filed on Jun. 29, 2004.
(60) Provisional application No. 60/669,756, filed on Apr. 8, 2005.

(51) Int. Cl. A61K 31/53; C07D 253/02

(52) U.S. Cl. 514/242; 544/182

(57) ABSTRACT

The present invention relates to methods of preparing compounds of formula I 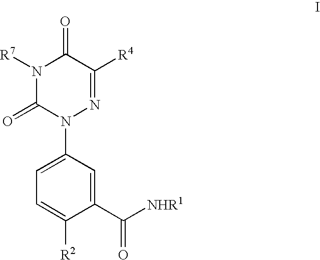
or a pharmaceutically acceptable salt thereof, wherein R¹, R², R⁴, and R⁷ have any of the values as defined in the specification. The compounds are useful as agents in the treatment of diseases, including inflammatory diseases such as rheumatoid arthritis. Also provided are compositions of crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising less than 2.5% residual organic solvent, and methods for preparing said compositions. Further provided are methods for crystallizing 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide.

### CROSS-REFERENCE TO RELATED APPLICATIONS

This application claims priority from U.S. Provisional Patent Application No. 60/583,813 filed Jun. 29, 2004, and U.S. Provisional Patent Application No. 60/669,756 filed Apr. 8, 2005.

### BACKGROUND OF THE INVENTION

The P2X₇ purinergic receptor (previously known as P2Z receptor), which is a ligand-gated ion channel, is present on a variety of cell types, largely those known to be involved in inflammatory/immune process, specifically, macrophages, mast cells and lymphocytes (T and B). Activation of the P2X₇ receptor by extracellular nucleotides, in particular adenosine triphosphate, leads to the release of interleukin-1β(IL-1β) and giant cell formation (macrophages/microglial cells), degranulation (mast cells) and proliferation (T cells), apoptosis, and L-selectin shedding (lymphocytes). P2X₇ receptors are also located on antigen-presenting cells (APC), keratinocytes, salivary acinar cells (parotid cells), hepatocytes and mesangial cells.

P2X₇ antagonists are known in the art, such as those described in International Patent Publications WO 01/46200, WO 01/42194, WO 01/44213, WO99/29660, WO 00/61569, WO 99/29661, WO 99/29686, WO 00/71529, and WO 01/44170, as well as in WO2003/042191.

Benzamides, heteroarylamides and reverse amides for uses other than inhibition of the P2X₇ receptor are described in various publications, such as International Patent Publications WO 97/22600, EP 138,527, WO 00/71509, WO 98/28269, WO 99/17777 and WO 01/58883.

Antagonists of the P2X₇ receptor are being identified for the treatment of human disease (see e.g., Alcaraz et al. (2003) Bioorg Med Chem Lett. 13(22):4043-4046; Baxter et al. (2003) Bioorg Med Chem Lett. 13(22):4047-4050). There is a need for additional compositions, and methods of preparing compounds that can inhibit the P2X₇ receptor for use as pharmaceutical agents.

### SUMMARY OF THE INVENTION

In one aspect, the present invention provides for methods of preparing a compound of formula I 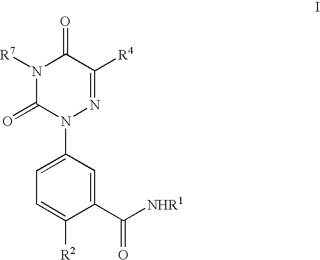
or a pharmaceutically acceptable salt thereof,
wherein R¹ is (C₁-C₆)alkyl, optionally substituted by (C₃-C₈)cycloalkyl, phenyl, naphthyl, 5 or 6-membered heterocycloalkyl, or a 5- or 6-membered heteroaryl, wherein each of said (C₁-C₆)alkyl, (C₃-C₈)cycloalkyl, phenyl, naphthyl, 5 or 6-membered heterocycloalkyl, or 5- or 6-membered heteroaryl are optionally substituted by one to three moieties independently selected from the group consisting of hydroxy, halo, CN—, (C₁-C₆)alkyl, HO(C₁-C₆)alkyl, (C₁-C₆)alkyl-NH(C═O)—, NH₂(C═O)—, (C₁-C₆)alkoxy, or (C₃-C₈)cycloalkyl;
R² is hydrogen, halo, —CN, or (C₁-C₆)alkyl, wherein said (C₁-C₆)alkyl is optionally substituted by one to three moieties, independently selected from the group consisting of halo, hydroxy, amino, —CN, (C₁-C₆)alkyl, (C₁-C₆)alkoxy, —CF₃, CF₃O—, (C₁-C₆)alkyl-NH—, [(C₁-C₆)alkyl]₂—N—, (C₁-C₆)alkyl-S—, (C₁-C₆)alkyl-(S═O)—, (C₁-C₆)alkyl-(SO₂)—, (C₁-C₆)alkyl-O—(C═O)—, formyl, (C₁-C₆)alkyl-(C═O)—, and (C₃-C₆)cycloalkyl;
wherein R⁴ is independently selected from the group consisting of hydrogen, halo, hydroxy, —CN, HO—(C₁-C₆)alkyl, (C₁-C₆)alkyl optionally substituted with one to three fluoro, (C₁-C₆)alkoxy optionally substituted with one to three fluoro, HO₂C—, (C₁-C₆)alkyl-O—(C═O)—, R⁵R⁶N(O₂S)—, (C₁-C₆)alkyl-(O₂S)—NH—, (C₁-C₆)alkyl-O₂S—[(C₁-C₆)alkyl-N]—, R⁵R⁶N(C═O)—, R⁵R⁶N(CH₂)ₘ—, phenyl, naphthyl, (C₃-C₈)cycloalkyl, 5- or 6-membered heteroaryl, 5 or 6-membered heterocycloalkyl, phenyl-O—, naphthyl-O—, (C₃-C₈)cycloalkyl-O—, 5- or 6-membered heteroaryloxy and 5 or 6-membered heterocycloalkyl-O—; and
R⁷ is —CH₂—C(R¹⁰R¹¹)—OH, wherein R¹⁰ and R¹¹ are independently selected from the group consisting of:
   hydrogen, phenyl, and (C₁-C₆)alkyl optionally substituted with one to three halos, hydroxy, —CN, (C₁-C₆)alkoxy-, ((C₁-C₆)alkyl), —N—, (C₁-C₆)alkyl-(C═O)—, (C₃-C₈)cycloalkyl-(C═O)—, 5 or 6-membered heterocycloalkyl-(C═O)—, phenyl-(C═O)—, naphthyl-(C═O)—, 5- or 6-membered heteroaryl-(C═O)—, (C₁-C₆)alkyl-(C═O)O—, (C₁-C₆)alkyl-O(C═O)—, (C₃-C₈)cycloalkyl, phenyl, naphthyl, 5 or 6-membered heterocycloalkyl, and 5- or 6-membered heteroaryl;
R⁵ and R⁶ are each independently selected from the group consisting of hydrogen, (C₁-C₆)alkyl, HO—(C₂-C₆)alkyl and (C₃-C₈)cycloalkyl, or R⁵ and R⁶ may optionally be taken together with the nitrogen atom to which they are attached to form a 5 or 6-membered heterocycloalkyl;
n is one or two; and
m is one or two;
wherein said method comprises reacting a compound of formula II 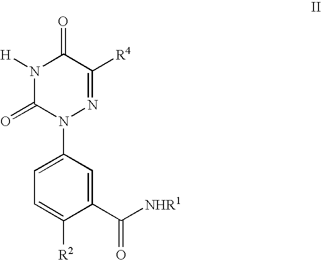
   with a compound of Formula VIII 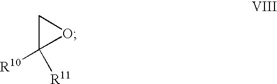
      in the presence of at least one Lewis acid. In certain embodiments the Lewis acid is an inorganic Lewis acid. In other embodiments the Lewis acid is boron trifluoride diethyl etherate. In still other embodiments, the Lewis acid is Al₂O₃, Ti(O-Prⁱ)₄, LiClO₄, or Zn(OAc)₂. In yet another embodiment, the Lewis acid is selected from (a) Eu(OTf)₃, Dy(OTf)₃, Ho(OTf)₃, Er(OTf)₃, Lu(OTf)₃, Yb(OTf)₃, Nd(OTf)₃, Gd(OTf)₃, Lu(OTf)₃, La(OTf)₃, Pr(OTf)₃, Tm(OTf)₃, Sc(OTf)₃, Sm(OTf)₃, AgOTf, or Y(OTf)₃; (b) AlCl₃, AlI₃, AlF₃, AlBr₃, AsCl₃, AsI₃, AsF₃, AsBr₃, BCl₃, BBr₃, BI₃, BF₃, FeCl₃, FeBr₃, FeI₃, FeF₃, FeCl₂, FeBr₂, FeI₂, FeF₂, GaCl₃, GaI₃, GaF₃, GaBr₃, MgCl₂, MgI₂, MgF₂, MgBr₂, NbCl₅, SbCl₃, SbI₃, SbF₃, SbBr₃, SbCl₅, SbI₅, SbF₅, SbBr₅, SnCl₂, SnI₂, SnF₂, SnBr₂, SnCl₄, SnL₄, SnF₄, SnBr₄, TiBr₄, TiCl₂, TiCl₃, TiCl₄, TiF₃, TiF₄, TiI₄, ZnCl₂, ZnI₂, ZnF₂, or ZnBr₂; (c) BF₃BCl₃-SMe₂, BI₃-SMe₂, BF₃—SMe₂, BBr₃-SMe₂, BF₃-OEt₂, Et₂AlCl, EtAlCl₂, MgCl₂.OEt₂, MgI₂-OEt₂, MgF₂-OEt₂, MgBr₂-OEt₂, Et₂AlCl, EtAlCl₂, or Zn(OAc)₂; and (d) (CH₃CO₂)₂Co, CoBr₂, CoCl₂, CoF₂, CoI₂, Co(NO₃)₂, cobalt (II) triflate, cobalt (II) tosylate, (CH₃CO₂)₂Cu, CuBr₂, CuCl₂, CuF₂, CuI₂, Cu(NO₃)₂, copper (II) triflate, copper (II) tosylate, (CH₃CO₂)₂Ni, NiBr₂, NiCl₂, NiF₂, NiI₂, Ni(NO₃)₂, nickel (II) triflate, or nickel (II) tosylate. In still other embodiments, the Lewis acid is a silica gel. In certain embodiments, the reaction is carried out in N,N-dimethylformamide, N,N-dimethyl acetamide, or N-methylpyrrolidinone or mixtures thereof. In particular embodiments, less than 6 moles of a compound of formula VIII is present per 1 mole of a compound of formula II; less than 5 moles of a compound of formula VIII is present per 1 mole of a compound of formula II; or between 1 to 2 moles of a compound of formula VIII is present per 1 mole of a compound of formula II. In other embodiments, the compound of formula VIII is (R)-(−)-glycidyl methyl ether. In additional embodiments, the compound of formula II is 2-Chloro-5-(3,5-dioxo-4,5-dihydro-3H-[1,2,4]triazin-2-yl)-N-(1-hydroxy-cycloheptylmethyl)-benzamide. In particular embodiments, the compound of formula I is 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide. In certain embodiments are provided methods of preparing 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide wherein said method comprises reacting 2-Chloro-5-(3,5-dioxo-4,5-dihydro-3H-[1,2,4]triazin-2-yl)-N-(1-hydroxy-cycloheptylmethyl)-benzamide with (R)-(−)-glycidyl methyl ether in the presence of a silica gel, wherein the reaction is carried out in N,N-dimethylformamide, N,N-dimethyl acetamide, or N-methylpyrrolidinone or mixtures thereof. The method also includes embodiments where R¹ is a (C₁-C₄)alkyl, optionally substituted by (C₃-C₈)cycloalkyl; wherein said (C₁-C₄)alkyl or (C₃-C₈)cycloalkyl are optionally substituted by one to three moieties independently selected from the group consisting of hydroxy, halo, CN—, (C₁-C₆)alkyl, HO(C₁-C₆)alkyl, (C₁-C₆)alkyl-NH(C═O)—, NH₂(C═O)—, (C₁-C₆)alkoxy, or (C₃-C₈)cycloalkyl. R² may be chloro, methyl or ethyl in certain embodiments. R⁴ is hydrogen and R⁷ is —CH₂—C(R¹⁰R¹¹)—OH, wherein R¹⁰ and R¹¹ may be independently selected from the group consisting of: hydrogen and (C₁-C₆)alkyl optionally substituted with (C₁-C₆)alkoxy- or —OH, in other embodiments. R⁷ may be selected from the group consisting of: 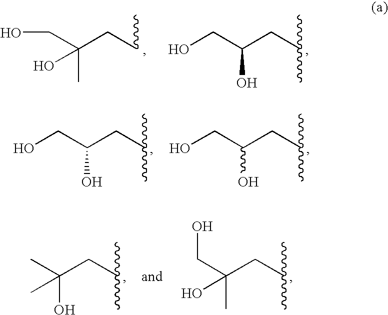
      in still other embodiments. R⁷ may be selected from the group consisting of:

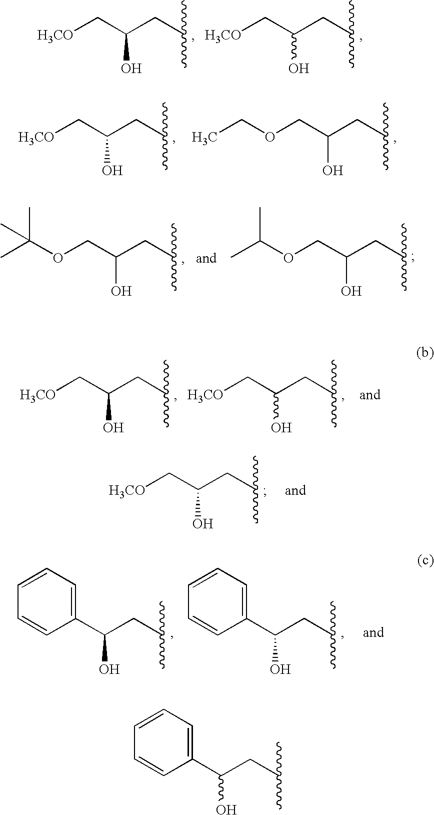

In another aspect, the present invention provides for compositions of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising: crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide; and less than 2.5% residual organic solvent. In certain embodiments, the compositions comprise less than 2.0% (w/w) residual organic solvent; between 0.1 and 2.0% (w/w) residual organic solvent; between 0.1 and 0.5% (w/w) residual organic solvent; or between 0.05 and 0.5% (w/w) residual organic solvent. In certain embodiments, the residual organic solvent is acetone. In particular embodiments, the composition has a melting point onset of between 108° C.±0.5 and 112° C.±0.5 as measured by Differential Scanning Calorimetry. In particular embodiments, the composition has a melting point onset of between 110° C.±0.5 and 112° C.±0.5 as measured by Differential Scanning Calorimetry. In certain embodiments, the composition has an X-ray powder diffraction comprising the following 2-theta values+0.2 measured using CuK_{α} radiation: 8.1, 16.4, 19.7, 21.2, 22.2, and 27.1. In still other embodiments, the composition has an X-ray powder diffraction comprising the following 2-theta values±0.2 measured using CuK_{α} radiation: 8.1, 11.7, 14.9, 16.4, 18.3, 19.7, 21.2, 21.6, 22.2, 22.6, and 27.1. In additional embodiments, the composition has an X-ray powder diffraction comprising the following 2-theta values±0.2 measured using CuK_{α} radiation: 7.8, 8.1, 10.5, 11.7, 13.2, 13.7, 14.3, 14.9, 15.6, 16.4, 17.3, 17.7, 18.3, 18.9, 19.1, 19.7, 20.3, 20.9, 21.2, 21.6, 22.2, 22.6, 22.8, 23.3, 23.9, 24.3, 24.6, 25.1, 25.9, 26.2, 27.1, 27.6, 28.2, 28.7, 28.8, 29.4, 30.0, 30.3, 30.9, 31.1, 31.9, 33.4, 33.8, 34.3, 35.2, and 37.1. In certain embodiments compositions may also be characterized by a solid-state ¹³C nuclear magnetic resonance comprising the following chemical shift differences between the lowest ppm resonance and other resonances: 150.6, 137.6, 119.5, and 54.8. In certain embodiments, the composition is characterized by a solid-state ¹³C nuclear magnetic resonance comprising the following chemical shift differences between the lowest ppm resonance and other resonances: 150.6, 137.6, 130.1, 129.2, 121.4, 120.5, 119.5, 117.7, 113, 112.7, 111.6, 110.3, 109.5, 107.3, 106, 54.8, 53.9, 47.7, 45.9, 41.2, 38, 34.2, 31.2, 24.7, 20.8, 19.0, 18.1, 17.4, 12.2, 10.1, 4.0, 3.5, and 1.2. In certain embodiments, the composition is characterized by a solid-state ¹³C nuclear magnetic resonance comprising the following chemical shifts expressed in parts per million: 169.8, 156.8, 138.7, and 74.0. In certain embodiments, the composition is characterized by a solid-state ¹³C nuclear magnetic resonance comprising the following chemical shifts expressed in parts per million: 169.8, 156.8, 149.3, 148.4, 140.6, 139.7, 138.7, 136.9, 132.2, 131.9, 130.8, 129.5, 128.7, 126.5, 125.2, 74.0, 73.1, 66.9, 65.1, 60.4, 57.2, 53.4, 50.4, 43.9, 40.0, 38.2, 37.3, 36.6, 31.4, 29.3, 23.2, 22.7, 20.4, and 19.2.

In another aspect, the present invention provides for processes for preparing a composition of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising less than 2.5% residual organic solvent comprising: combining n-heptane with a solution of acetone comprising 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide to generate crystals of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide; and isolating crystals of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising less than 2.5% (w/w) residual organic solvent. In certain embodiments, isolating comprises comprises filtering crystals from the solvent, and drying the crystals. In other embodiments, the composition has less than 2.0% (w/w) residual organic solvent. In still other embodiments, the composition has between 0.1 and 2.0% (w/w) residual organic solvent. In yet other embodiments, the composition has between 0.1 and 0.5% (w/w) residual organic solvent. The residual organic solvent may be acetone. In particular embodiments, the composition has a melting point onset of between 108° C.±0.5 and 112° C.±0.5 as measured by Differential Scanning Calorimetry. In particular embodiments, the composition has a melting point onset of between 110° C.±0.5 and 112° C.±0.5 as measured by Differential Scanning Calorimetry. In certain embodiments, the composition has an X-ray powder diffraction comprising the following 2-theta values±0.2 measured using CuK_{α} radiation: 8.1, 16.4, 19.7, 21.2, 22.2, and 27.1. In still other embodiments, the composition has an X-ray powder diffraction comprising the following 2-theta values±0.2 measured using CuK_{α} radiation: 8.1, 11.7, 14.9, 16.4, 18.3, 19.7, 21.2, 21.6, 22.2, 22.6, and 27.1. In additional embodiments, the composition has an X-ray powder diffraction comprising the following 2-theta values±0.2 measured using CuK_{α} radiation: 7.8, 8.1, 10.5, 11.7, 13.2, 13.7, 14.3, 14.9, 15.6, 16.4, 17.3, 17.7, 18.3, 18.9, 19.1, 19.7, 20.3, 20.9, 21.2, 21.6, 22.2, 22.6, 22.8, 23.3, 23.9, 24.3, 24.6, 25.1, 25.9, 26.2, 27.1, 27.6, 28.2, 28.7, 28.8, 29.4, 30.0, 30.3, 30.9, 31.1, 31.9, 33.4, 33.8, 34.3, 35.2, and 37.1. In certain embodiments, compositions may also be characterized by a solid-state ¹³C nuclear magnetic resonance comprising the following chemical shift differences between the lowest ppm resonance and other resonances: 150.6, 137.6, 119.5, and 54.8. In certain embodiments, the composition is characterized by a solid-state 1³C nuclear magnetic resonance comprising the following chemical shift differences between the lowest ppm resonance and other resonances: 150.6, 137.6, 130.1, 129.2, 121.4, 120.5, 119.5, 117.7, 113, 112.7, 111.6, 110.3, 109.5, 107.3, 106, 54.8, 53.9, 47.7, 45.9, 41.2, 38, 34.2, 31.2, 24.7, 20.8, 19.0, 18.1, 17.4, 12.2, 10.1, 4.0, 3.5, and 1.2. In certain embodiments, the composition is characterized by a solid-state ¹³C nuclear magnetic resonance comprising the following chemical shifts expressed in parts per million: 169.8, 156.8, 138.7, and 74.0. In certain embodiments, the composition is characterized by a solid-state ¹³C nuclear magnetic resonance comprising the following chemical shifts expressed in parts per million: 169.8, 156.8, 149.3, 148.4, 140.6, 139.7, 138.7, 136.9, 132.2, 131.9, 130.8, 129.5, 128.7, 126.5, 125.2, 74.0, 73.1, 66.9, 65.1, 60.4, 57.2, 53.4, 50.4, 43.9, 40.0, 38.2, 37.3, 36.6, 31.4, 29.3, 23.2, 22.7, 20.4, and 19.2.

In another aspect, the invention relates to processes for preparing a composition of crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising less than 2.5% residual organic solvent comprising: combining n-heptane with a solution of acetone comprising 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide to generate crystals of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide; and isolating crystals of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising less than 2.5% (w/w) residual organic solvent. In certain embodiments, the process comprises filtering crystals from the solvent, and drying the crystals. In certain embodiments, the composition has less than 2.0% (w/w) residual organic solvent; between 0.1 and 2.0% (w/w) residual organic solvent; or between 0.1 and 0.5% (w/w) residual organic solvent.

In another aspect, the present invention provides for methods of treating a subject suffering from a disease selected from the group consisting of rheumatoid arthritis, ankylosing spondylitis, osteoarthritis, psoriatic arthritis, psoriasis, inflammatory diseases, and autoimmune diseases, the method comprising: administering a therapeutically effective amount of a composition comprising crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising less than 2.5% residual organic solvent. In certain embodiments, the disease is rheumatoid arthritis. The disease may also be an IL-1 mediated disease. As defined herein, a “IL-1 mediated disease” includes but is not limited to a disease or disorder selected from the group consisting of arthritis (including psoriatic arthritis, Reiter's syndrome, rheumatoid arthritis, gout, traumatic arthritis, rubella arthritis, rheumatoid spondylitis, osteoarthritis, gouty arthritis and acute synovitis), inflammatory bowel disease, Crohn's disease, emphysema, acute respiratory distress syndrome, adult respiratory distress syndrome, asthma, bronchitis chronic obstructive pulmonary disease, chronic pulmonary inflammatory disease, silicosis, pulmonary sarcoidosis, allergic reactions, allergic contact hypersensitivity, eczema, contact dermatitis, psoriasis, sunburn, cancer, tissue ulceration, restenosis, periodontal disease, epidermolysis bullosa, osteoporosis, bone resorption disease, loosening of artificial joint implants, atherosclerosis, aortic aneurysm, congestive heart failure, myocardial infarction, stroke, cerebral ischemia, head trauma, neurotrauma, spinal cord injury, neuro-degenerative disorders, Alzheimer's disease, Parkinson's disease, migraine, depression, peripheral neuropathy, pain, cerebral amyloid angiopathy, nootropic or cognition enhancement, amyotrophic lateral sclerosis, multiple sclerosis, ocular angiogenesis, corneal injury, macular degeneration, corneal scarring, scleritis, abnormal wound healing, burns, autoimmune disorders, Huntington's disease, diabetes, AIDS, cachexia, sepsis, septic shock, endotoxic shock, conjunctivitis shock, gram negative sepsis, toxic shock syndrome, cerebral malaria, cardiac and renal reperfusion injury, thrombosis, glomerularonephritis, graft vs. host reaction, allograft rejection, organ transplant toxicity, ulcerative colitis, or muscle degeneration.

In another aspect, the present invention provides for pharmaceutical compositions comprising: a therapeutically effective amount of a crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising less than 2.5% residual organic solvent admixed with at least one pharmaceutically acceptable carrier.

In another aspect, the present invention provides for processes for preparing a pharmaceutical composition comprising:
admixing crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising less than 2.5% residual organic solvent with at least one pharmaceutically acceptable carrier.

In another aspect, the present invention provides for compositions of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising:
crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide; having a melting point onset of between 108° C.±0.5 and 112° C.±0.5 as measured by Differential Scanning Calorimetry. In certain embodiments, the composition has a melting point onset of between 110° C.±0.5 and 112° C.±0.5 as measured by Differential Scanning Calorimetry.

In another aspect, the present invention provides for processes for preparing a pharmaceutical compositions comprising: admixing a 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide having a melting point onset of between 108° C.±0.5 and 112° C.±0.5 as measured by Differential Scanning Calorimetry with at least one pharmaceutically acceptable carrier.

In another aspect, the present invention provides for processes for preparing crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising: crystallizing 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide from a solution of acetone, diisopropyl ether, n-butyl acetate, n-heptane, methanol, tetrahydrofuran, or methylethyl ketone.

The compounds of this invention include all stereoisomers (e.g., cis and trans isomers) and all optical isomers of compounds of formula I (e.g., R and S enantiomers), as well as racemic, diastereomeric and other mixtures of such isomers.

The compounds, and salts of the present invention can exist in several tautomeric forms, including the enol and imine form, and the keto and enamine form and geometric isomers and mixtures thereof. All such tautomeric forms are included within the scope of the present invention. Tautomers exist as mixtures of a tautomeric set in solution. In solid form, usually one tautomer predominates. Even though one tautomer may be described, the present invention includes all tautomers of the present compounds. One example of a tautomeric structure is a group of:

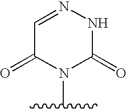

One skilled in the art will appreciate that this group can also be drawn as its tautomer:

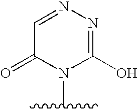

The present invention also includes atropisomers. Atropisomers refer to compounds of formula I that can be separated into rotationally restricted isomers.

The compounds of this invention may contain olefin-like double bonds. When such bonds are present, the compounds of the invention exist as cis and trans configurations and as mixtures thereof.

### DEFINITIONS

The term “alkyl group” or “alkyl” includes straight and branched carbon chain radicals. The term “alkylene” refers to a diradical of an unsubstituted or substituted alkane. For example, a “C₂₋₆ alkyl” is an alkyl group having from 2 to 6 carbon atoms. Examples of C₂-C₆ straight-chain alkyl groups include, but are not limited to, ethyl, n-propyl, n-butyl, n-pentyl, and n-hexyl. Examples of branched-chain alkyl groups include, but are not limited to, isopropyl, tert-butyl, isobutyl, etc. Examples of alkylene groups include, but are not limited to, —CH₂—, —CH₂—CH₂−, —CH₂—CH(CH₃)—CH₂—, and —(CH₂)₁₋₃. Alkylene groups can be substituted with groups as set forth below for alkyl.

The term alkyl includes both “unsubstituted alkyls” and “substituted alkyls,” the latter of which refers to alkyl moieties having substituents replacing a hydrogen on one or more carbons of the hydrocarbon backbone. Such substituents are independently selected from the group consisting of: halo, I, Br, Cl, F, —OH, —COOH, trifluoromethyl, —NH₂, —OCF₃, and O—C₁-C₃.

Typical substituted alkyl groups thus are 2,3-dichloropentyl, 3-hydroxy-5-carboxyhexyl, 2-aminopropyl, pentachlorobutyl, trifluoromethyl, methoxyethyl, 3-hydroxypentyl, 4-chlorobutyl, 1,2-dimethyl-propyl, and pentafluoroethyl.

“Halo” includes fluoro, chloro, bromo, and iodo.

The term “C₃-C₈cycloalkyl” refers to a cycloalkyl group containing from 3 to 8 carbons. Thus, the term “C₃-C₈cycloalkyl” encompasses monocyclic cycloalkyl groups containing from 3 to 8 carbons and bicyclic cycloalkyl groups containing 7 or 8 carbons. Examples of “C₃-C₈cycloalkyls” include, but are not limited to, cyclopropyl, cyclobutyl, cyclopentyl, cyclohexyl, cycloheptyl, and bicyclo[2.2.1]heptyl; the cycloalkyl group may optionally contain 1 or 2 double bonds (i.e., a cycloalkylenyl) including, but not limited to, cyclopentenyl, cyclohexenyl, and cycloheptenyl. A “C₃-C₈cycloalkyl” may be substituted with 1 or 2 groups independently selected from C₁-C₃alkyl (e.g., methyl) and —O—C₁-C₃alkyl (e.g., methoxy). Examples of substituted cycloalkyl groups include, but are not limited to, methyl-cyclopropyl, dimethyl-cyclohexyl, 2-methyl-cyclohexyl, 3-methyl-cyclohexyl, 3,5-dimethyl-cyclohexyl, and 4-methyl-cyclohexyl.

A “5-membered heterocycloalkyl” is a stable 5-membered, monocyclic cycloalkyl ring having from 2 to 4 carbon atoms and from 1 to 3 heteroatoms selected from the group consisting of: 1 O; 1 S; 1 N; 2 N; 3 N; 1 S and 1 N; 1 S, and 2 N; 1 O and 1 N; and 1 O and 2 N. Illustrative examples of stable 5-membered heterocycloalkyls include tetrahydrofuranyl, dihydrofuranyl, tetrahydrothienyl, dihydrothienyl, imidazolidinyl, oxazolidinyl, imidazolinyl, isoxazolidinyl, pyrrolidinyl, 2-pyrrolinyl, and 3-pyrrolinyl.

A “6-membered heterocycloalkyl” is a stable 6-membered, monocyclic cycloalkyl ring having from 3 to 5 carbon atoms and from 1 to 3 heteroatoms selected from the group consisting of: 1 O; 2 O;1 S; 2 S; 1 N; 2 N; 3 N; 1 S, 1 O, and 1 N; 1 S and 1 N; 1 S and 2 N; 1 S and 10; 1 S and 2 O; 1 O and 1 N; and 1 O and 2 N. Illustrative examples of stable 6-membered heterocycloalkyls include tetrahydropyranyl, dihydropyranyl, dioxanyl, 1,3-dioxolanyl, 1,4-dithianyl, hexahydropyrimidine, morpholinyl, piperazinyl, piperidinyl, 2H-pyranyl, 4H-pyranyl, pyrazolidinyl, pyrazolinyl, 1,2,3,6-tetrahydropyridinyl, tetrahydrothiopyranyl, 1,1-dioxo-hexahydro-1λ⁶-thiopyranyl, 1,1-dioxo-1λ⁶-thiomorpholinyl, thiomorpholinyl, thioxanyl, and trithianyl.

The foregoing heterocycloalkyls can be C-attached or N-attached. For example, piperidinyl can be piperidin-1-yl (N-attached) or piperidin-4-yl (C-attached).

Embraced within the term “5 or 6 membered heterocycloalkyl” are 5 membered rings having one carbon-carbon or one carbon-nitrogen double bond in the ring (e.g., 2-pyrrolinyl, 3-pyrrolinyl, etc.) and 6 membered rings having one carbon-carbon or one carbon-nitrogen double bond in the ring (e.g., dihydro-2H-pyranyl, 1,2,3,4-tetrahydropyridine, 3,4-dihydro-2H-[1,4]oxazine, etc.). “5 or 6-membered heterocycloalkyls” may be substituted such as those set out above for C₃-C₈cycloalkyls, where possible.

The term “phenyl” refers to unsubstituted and substituted phenyl groups. A phenyl group may be substituted with 1 to 3 substituents independently selected from the group consisting of: C₁-C₃alkyl, —O—C₁-C₃alkyl, —OCF₃, halo, and a C₅-C₆ cycloalkyl.

Typical substituted phenyl groups include, but are not limited to, 3-chlorophenyl, 2,6-dibromophenyl, 2,4,6-tribromophenyl, 2,6-dichlorophenyl, 4-trifluoromethylphenyl, 3-methyl-phenyl, 4-methyl-phenyl, 3,5-dimethyl-phenyl, 3,4,5-trimethoxy-phenyl, 3,5-dimethoxy-phenyl, 3,4-dimethoxy-phenyl, 3-methoxy-phenyl, 4-methoxy-phenyl, 3,5-difluoro-phenyl, 4-chloro-phenyl, 3-trifluoromethyl-phenyl, 3,5-dichloro-phenyl, 2-methoxy-5-methyl-phenyl, 2-fluoro-5-methyl-phenyl, 4-chloro-2-trifluoromethyl-phenyl, and the like.

A “5-membered heteroaryl” is a stable 5-membered, monocyclic, aromatic ring radial having from 1 to 4 carbon atoms and from 1 to 4 heteroatoms selected from the group consisting of: 1 O; 1 S; 1 N; 2 N; 3 N; 4 N; 1 S and 1 N; 1 S and 2 N; 1 O and 1 N; and 1 O and 2 N. Illustrative examples of stable 5-membered heteroaryls include, but are not limited to, furanyl, 2-furanyl, 3-furanyl, imidazolyl, isoxazolyl, isothiazolyl, oxadiazolyl, oxazolyl, pyridinyl, 2-, 3-, or 4-pyridinyl, pyrimidinyl, 2-, 4-, or 5-pyrimidinyl, pyrazolyl, pyrrolyl, 2- or 3-pyrrolyl, pyrazinyl, pyridazinyl, 3- or 4-pyridazinyl, 2-pyrazinyl, thienyl, 2-thienyl, 3-thienyl, tetrazolyl, thiazolyl, thiadiazolyl, triazinyl and triazolyl.

A “6-membered heteroaryl” is a stable 6-membered, monocyclic, aromatic ring radical having from 3 to 5 carbon atoms and from 1 to 3 heteroatoms selected from the group consisting of: 1 N; 2 N; and 3 N. Illustrative examples of stable 6-membered heteroaryl include pyridin-2-yl, pyridin-4-yl, pyrimidin-2-yl, pyridazin-4-yl, and pyrazin-2-yl.

A 5- or 6-membered heteroaryl group may be optionally substituted with 1 to 3 substituents independently selected from the group consisting of: C₁-C₃alkyl, —O—C₁-C₃alkyl, —OCF₃, and halo.

A “naphthyl group” refers to unsubstituted and substituted naphthyl groups. A naphthyl group may be substituted with 1 to 4 substituents independently selected from the group consisting of: C₁-C₃alkyl, —O—C₁-C₃alkyl, —OCF₃, halo, and a C₅-C₆ cycloalkyl.

### BRIEF DESCRIPTION OF THE DRAWINGS

FIG. 1

Differential Scanning Calorimetry thermal profile of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide (Example 4, Sample D).

FIG. 2

Solid-State ¹³C-NMR spectrum of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide (Example 4, Sample D).

FIG. 3

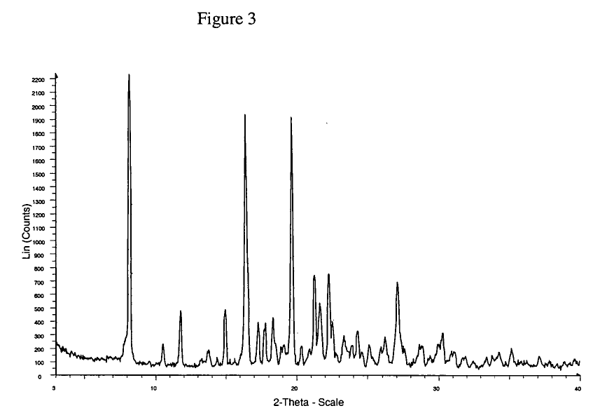

Powder X-ray Diffraction (PXRD) spectrum of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide (Example 4, Sample D).

### DETAILED DESCRIPTION OF THE INVENTION

### I. Introduction

The present invention relates to the preparation of compounds of Formula I and pharmaceutically acceptable salts thereof, that are useful as agents in the treatment of diseases, including inflammatory diseases such as rheumatoid arthritis. Also provided are compositions of crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising less than 2.5% residual organic solvent, and methods for preparing said compositions. Further provided are methods for crystallizing 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide.

### II. Process of Preparing Compounds

Compounds of Formula I can be prepared by applying synthetic methodology known in the art and synthetic methodology outlined in the schemes set forth below as well as using methods described in U.S. patent application Ser. No. 10/748,340, which is hereby incorporated by reference in its entirety.

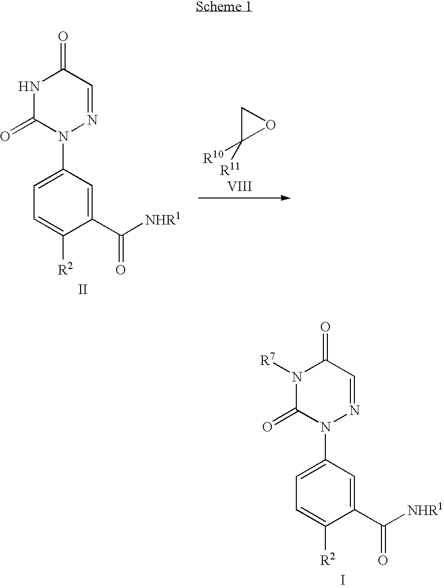

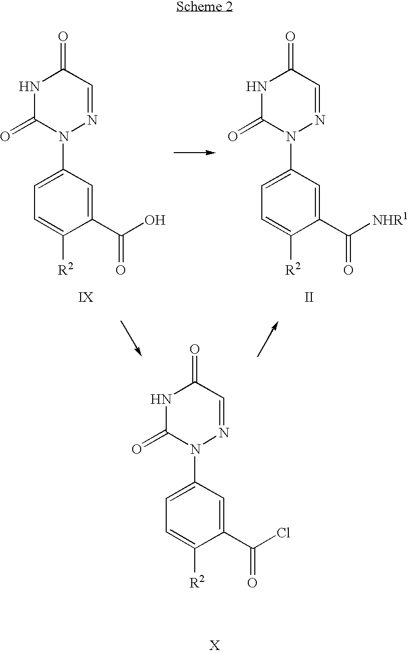

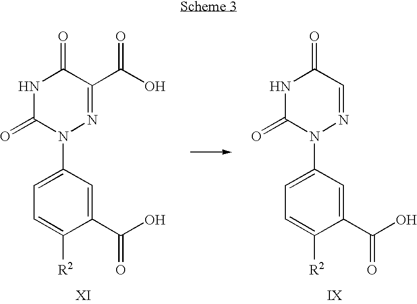

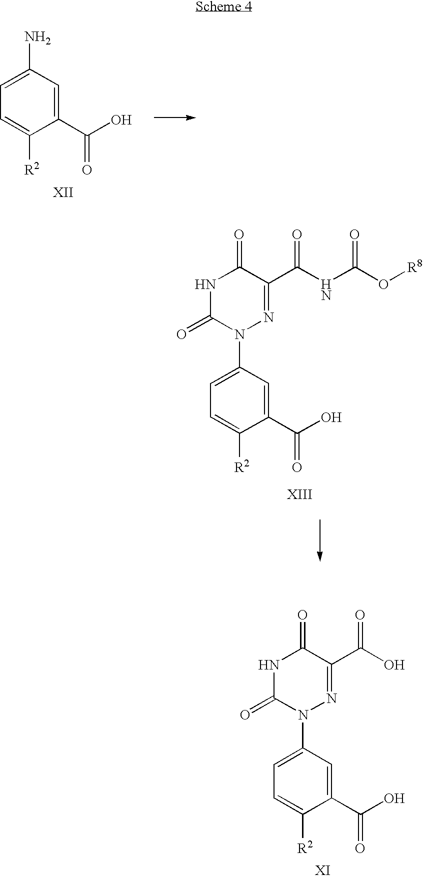

Scheme 1 refers to the preparation of compounds of formula I. Compounds of formula I (e.g., 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide) can be prepared from compounds of formula II (e.g., 2-Chloro-5-(3,5-dioxo-4,5-dihydro-3H-[1,2,4]triazin-2-yl)-N-(1-hydroxy-cycloheptylmethyl)-benzamide) by reaction with an appropriately substituted oxirane (e.g., (R)-(−)-glycidyl methyl ether) of formula VIII in the presence of a catalytically effective amount of a Lewis acid, and a polar solvent including but not limited to N,N-dimethylformamide, N,N-dimethyl acetamide, or N-methylpyrrolidinone, dimethylsulfoxide, and tetrahydrofuran. The aforesaid reaction can be performed at temperatures ranging from 0° C. to 100° C. for a period of 2 to 72 hours, where the preferred conditions are dimethylformamide at 60° C. for 24 hours. In certain embodiments, the reaction can be carried out under inert reaction conditions using an inert solvent (e.g., an anhydrous solvent) under an inert gas atmosphere (e.g., nitrogen gas). Examples of Lewis acids include compounds having the formula MXₜ, where M is selected from the group containing Al, As, B, Fe, Fe, Ga, Mg, Nb, Sb, Sn, Ti, and Zn. X is a halide selected from the group consisting of Cl, I, F, and Br. Those of skill in the art will recognize that t is an integer from 2 to 5 depending on the valence state of M. Examples of compounds of formula MXₜ include, but are not limited to: AlCl₃, AlI₃, AlF₃, AlBr₃, AsCl₃, AsI₃, AsF₃, AsBr₃, BCl₃, BBr₃, BI₃, BF₃, FeCl₃, FeBr₃, FeI₃, FeF₃, FeCl₂, FeBr₂, FeI₂, FeF₂, GaCl₃, GaI₃, GaF₃, GaBr₃, MgCl₂, MgI₂, MgF₂, MgBr₂, NbCl₅, SbCl₃, SbI₃, SbF₃, SbBr₃, SbCl₅, SbI₅, SbF₅, SbBr₅, SnCl₂, SnI₂, SnF₂, SnBr₂, SnCl₄, SnI₄, SnF₄, SnBr₄, TiBr₄, TiCl₂, TiCl₃, TiCl₄, TiF₃, TiF₄, TiI₄, ZnCl₂, ZnI₂, ZnF₂, and ZnBr₂. In addition, Lewis acids such as Al₂O₃, BF₃BCl₃-SMe₂, BI₃-SMe₂, BF₃—SMe₂, BBr₃ SMe₂, BF₃—OEt₂, Et₂AlCl, EtAlCl₂, MgCl₂-OEt₂, MgI₂-OEt₂, MgF₂—OEt₂, MgBr₂-OEt₂, Et₂AlCl, EtAlCl₂, LiClO₄ (lithium perchlorate), Ti(O—Prⁱ)₄ (titanium tetraisopropoxide), and Zn(OAc)₂ may be employed. In another embodiment, Cobalt (II), Copper (II), and Nickel (II) salts such as (CH₃CO₂)₂Co, CoBr₂, CoCl₂, CoF₂, CoI₂, Co(NO₃)₂, cobalt (II) triflate, cobalt (II) tosylate, (CH₃CO₂)₂Cu, CuBr₂, CuCl₂, CuF₂, CuI₂, Cu(NO₃)₂, copper (II) triflate, copper (II) tosylate, (CH₃CO₂)₂Ni, NiBr₂, NiCl₂, NiF₂, NiI₂, Ni(NO₃)₂, nickel (II) triflate, and nickel (II) tosylate can be used in the reaction of VIII and II. Monoalkyl boronhalides, dialkyl boronhalides, monoaryl boronhalides, and diaryl boronhalides may be employed as Lewis acids. Rare earth metal trifluoromethanesulfonates such as Eu(OTf)₃, Dy(OTf)₃, Ho(OTf)₃, Er(OTf)₃, Lu(OTf)₃, Yb(OTf)₃, Nd(OTf)₃, Gd(OTf)₃, Lu(OTf)₃, La(OTf)₃, Pr(OTf)₃, Tm(OTf)₃, Sc(OTf)₃, Sm(OTf)₃, AgOTf, Y(OTf)₃, and polymer resins thereof (e.g., Scandium triflate polystyrene resin; PS—Sc(OTf)₂) can be used in a solution such as one part water and four to nine parts tetrahydrofuran. Furthermore, silica gels may be employed in the reaction such as silica gel (CAS 112926-00-8) used for column chromatography, preferably in the range of 80-500 mesh particle size. In certain embodiments, the Lewis Acid is a silica gel and the reaction is carried out in a solvent such as N,N-dimethylformamide, N,N-dimethyl acetamide, or N-methylpyrrolidinone, or mixtures thereof. The aforementioned Lewis acids also include heteropoly acids or their salts, zeolite-type molecular sieve, Lewis conjugate acid-type super acid, or Lewis acid (such as AlCl₃, BF₃, or XF₅ (X=P, As, Sb, or Bi))-treated oxide or molecular sieve, and loaded with porous inorganic carrier (such as activated C, SiO₂, Al₂O₃, MgO, TiO₂, natural or synthetic aluminosilicate-type zeolite).

Scheme 2 refers to the preparation of compounds of formula II. Compounds of formula II can be prepared from compounds of formula IX (e.g., 2-Chloro-5-(3,5-dioxo-4,5-dihydro-3H-[1,2,4]triazin-2-yl)-benzoic acid) by reacting with a compound of formula XIV, H₂N—R¹ (e.g., 1-aminomethyl-cycloheptanol HCl), in the presence of a coupling reagent such as 1-[3-(dimethylamino)propyl]-3-ethylcarbodiimide (EDCI), dicyclohexylcarbodiimide (DCC), 1,1′-carbonyldiimidazole (CDI) and a base such as dimethylaminopyridine (DMAP) or triethylamine in an aprotic solvent, such as methylene chloride, dimethylformamide, or dimethylsulfoxide, preferably 1-[3-(dimethylamino)propyl]-3-ethylcarbodiimide and dimethylaminopyridine in dimethyl formamide. The aforesaid reaction may be run at a temperature from 22° C. to 60° C., for a period of 1 hour to 20 hours, preferably 22° C. for 18 hours.

Compounds of formula V may also be prepared from compounds of formula X (e.g., 2-Chloro-5-(3,5-dioxo-4,5-dihydro-3H-[1,2,4]triazin-2-yl)-benzoyl chloride) by reaction by reacting with a compound of formula XIV in the presence of a base including but not limited to dimethylaminopyridine (DMAP), triethylamine, aqueous sodium hydroxide or aqueous potassium hydroxide in an aprotic solvent, such as methylene chloride, ethyl acetate, dichloroethane, dimethylformamide, or dimethylsulfoxide, preferably aqueous sodium hydroxide and dichloroethane. The aforesaid reaction may be run at a temperature from 22° C. to 60° C., for a period of 1 hour to 24 hours, preferably at ambient temperature for 3 hours.

Compound X can be prepared from compound IX by reaction with a reagent capable of generating an acid chloride such as thionyl chloride or oxalyl chloride in the presence of a polar aprotic solvent such as ethyl acetate, methylene chloride, or dichloroethane at a temperature of 22° C. to 60° C., for a period of 1 hour to 24 hours, preferably oxalyl chloride in methylene chloride at ambient temperature for 16 hours.

Scheme 3 refers to the preparation of compounds of formula IX, which can be converted into compounds of formula V by the methods described in Scheme 2. Compounds of formula IX can be prepared from compounds of formula XI using decarboxylation conditions, preferably mercaptoacetic acid in water containing a base such as sodium hydroxide at a temperature from 22° C. to 160° C. for a period of 1 hour to 24 hours, preferably 100° C. for 18 hours.

Scheme 4 refers to the preparation of compounds of formula XIII and XI, Compounds of formula XI can be converted into compounds of formula IX by the methods described in Scheme 3.

A compound of formula XI can be prepared from a compound of formula XIII, wherein R⁸ is a (C₁-C₂)alkyl, by reaction with an acid such as 50% sulfuric acid at a temperature between 60° C. and 120° C., generally for a period between 30 minutes and 6 hours, preferably 2 hours at 120° C.

A compound of formula XIII, wherein R⁸ is a (C₁-C₂)alkyl, can be prepared from the diazonium intermediate derived from a compound of formula XII. The diazonium intermediate is prepared by reaction of a compound of formula XII with an acid such as hydrochloric acid and/or glacial acetic acid, followed by treatment with sodium nitrite in a solvent such as water at a temperature from 0° C. to 25° C., and the reaction is generally run from a period of 30 minutes to about 2 hours, preferably 10° C. for 30 minutes. A compound of formula XII is prepared by the reaction of above diazonium intermediate with a compound of formula XVII: R⁸O(C═O)N(C═O)CH₂(C═O)N(C═O)OR⁸, under basic conditions. The reaction is typically carried out with sodium acetate as the base at a temperature from 0° C. to 120° C., preferably 10° C., then warmed to 120° C., and the reaction is generally run for a period of 1 hour to 24 hours, preferably 4 hours (Carrool et. al.; J. Med. Chem., 1983, 26, 96-100).

One of ordinary skill in the art will appreciate that in some cases protecting groups may be required during preparation. After the target molecule is made, the protecting group can be removed by methods well known to those of ordinary skill in the art, such as described in Greene and Wuts, “Protective Groups in Organic Synthesis” (3rd Ed, John Wiley & Sons 1999).

Examples of compounds which may be made using the foregoing schemes and description include those in Table 1:

TABLE 1

| # | STRUCTURE | NAME |
|---|---|---|
| 1 | 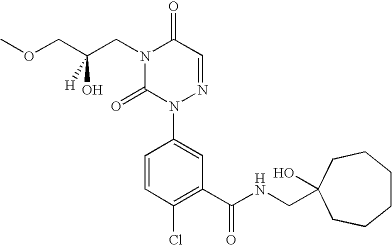 | 2-Chloro-N-(1-hydroxy- cycloheptylmethyl)-5-[4-(2- hydroxy-3-methoxy-propyl)-3,5- dioxo-4,5-dihydro-3H- [1,2,4]triazin-2-yl]-benzamide |
| 2 | 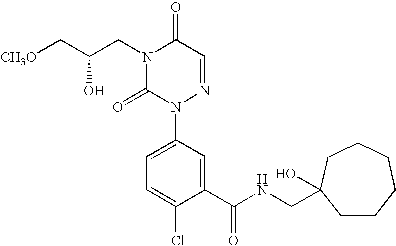 | 2-Chloro-N-(1-hydroxy- cycloheptylmethyl)-5-[4-(2- hydroxy-3-methoxy-propyl)-3,5- dioxo-4-dihydro-3H- [1,2,4]triazin-2-yl]-benzamide |
| 3 | 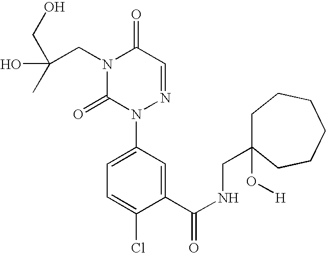 | 2-Chloro-5-[4-(2,3-dihydroxy-2- methyl-propyl)-3,5-dioxo-4,5- dihydro-3H-[1,2,4]triazin-2-yl]-N- (1-hydroxy-cycloheptylmethyl)- benzamide |
| 4 | 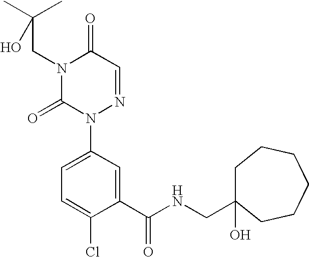 | 2-Chloro-N-(1-hydroxy- cycloheptylmethyl)-5-[4-(2- hydroxy-2-methyl-propyl)-3,5- dioxo-4,5-dihydro-3H- [1,2,4]triazin-2-yl]-benzamide |
| 5 | 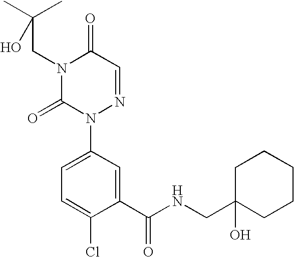 | 2-Chloro-N-(1-hydroxy- cyclohexylmethyl)-5-[4-(2-hydroxy- 2-methyl-propyl)-3,5-dioxo-4,5- dihydro-3H-[1,2,4]triazin-2-yl]- benzamide |
| 6 | 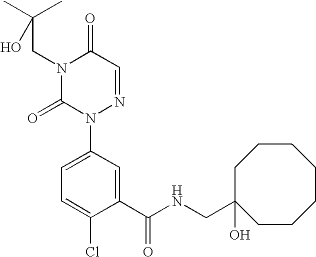 | 2-Chloro-N-(1-hydroxy- cyclooctylmethyl)-5-[4-(2-hydroxy- 2-methyl-propyl)-3,5-dioxo-4,5- dihydro-3H-[1,2,4]triazin-2-yl]- benzamide |
| 7 | 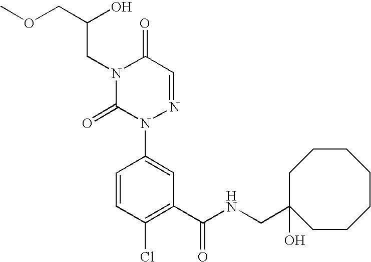 | 2-Chloro-N-(1-hydroxy- cyclooctylmethyl)-5-[4-(2-hydroxy- 3-methoxy-propyl)-3,5-dioxo-4,5- dihydro-3H-[1,2,4]triazin-2-yl]- benzamide |
| 8 | 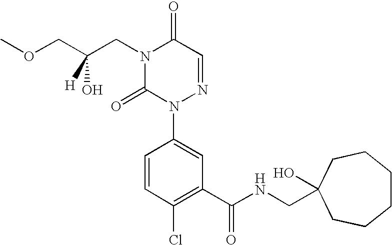 | 2-Chloro-N-(1-hydroxy- cycloheptylmethyl)-5-[4-(2- hydroxy-3-methoxy-propyl)-3,5- dioxo-4,5-dihydro-3H- [1,2,4]triazin-2-yl]-benzamide |
| 9 | 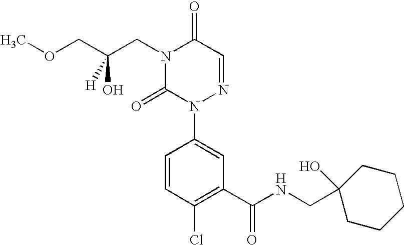 | 2-Chloro-N-(1-hydroxy- cyclohexylmethyl)-5-[4-(2-hydroxy- 3-methoxy-propyl)-3,5-dioxo-4,5- dihydro-3H-[1,2,4]triazin-2-yl]- benzamide |
| 10 | 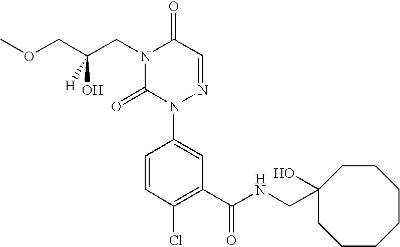 | 2-Chloro-N-(1-hydroxy- cyclooctylmethyl)-5-[4-(2-hydroxy- 3-methoxy-propyl)-3,5-dioxo-4,5- dihydro-3H-[1,2,4]triazin-2-yl]- benzamide |
| 11 | 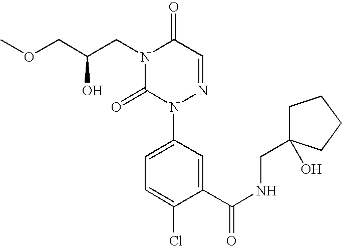 | 2-Chloro-N-(1-hydroxy- cyclopentylmethyl)-5-[4-(2- hydroxy-3-methoxy-propyl)-3,5- dioxo-4,5-dihydro-3H- [1,2,4]triazin-2-yl]-benzamide |
| 12 | 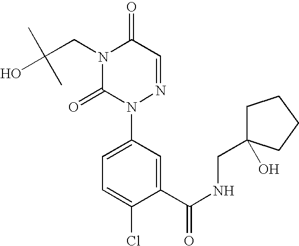 | 2-Chloro-N-(1-hydroxy- cyclopentylmethyl)-5-[4-(2- hydroxy-2-methyl-propyl)-3,5- dioxo-4,5-dihydro-3H- [1,2,4]triazin-2-yl]-benzamide |
| 13 | 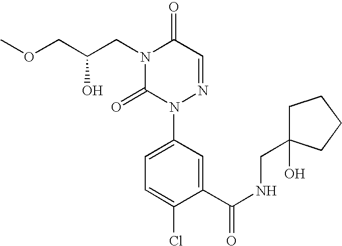 | 2-Chloro-N-(1-hydroxy- cyclopentylmethyl)-5-[4-(2- hydroxy-3-methoxy-propyl)-3,5- dioxo-4,5-dihydro-3H- [1,2,4]triazin-2-yl]-benzamide |
| 14 | 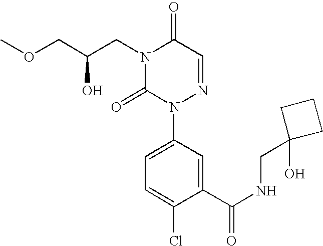 | 2-Chloro-N-(1-hydroxy- cyclobutylmethyl)-5-[4-(2-hydroxy- 3-methoxy-propyl)-3,5-dioxo-4,5- dihydro-3H-[1,2,4]triazin-2-yl]- benzamide |
| 15 | 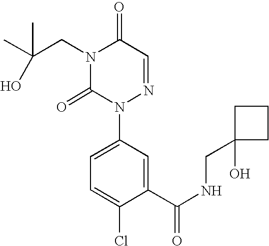 | 2-Chloro-N-(1-hydroxy- cyclobutylmethyl)-5-[4-(2-hydroxy- 2-methyl-propyl)-3,5-dioxo-4,5- dihydro-3H-[1,2,4]triazin-2-yl]- benzamide |
| 16 | 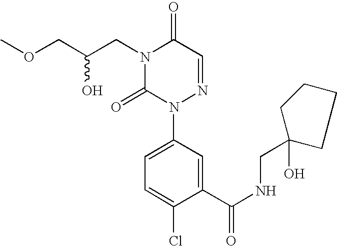 | 2-Chloro-N-(1-hydroxy- cyclopentylmethyl)-5-[4-(2- hydroxy-3-methoxy-propyl)-3,5- dioxo-4,5-dihydro-3H- [1,2,4]triazin-2-yl]-benzamide |
| 17 | 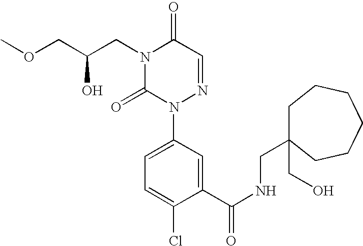 | 2-Chloro-5-[4-(2-hydroxy-3- methoxy-propyl)-3,5-dioxo-4,5- dihydro-3H-[1,2,4]triazin-2-yl]-N- (1-hydroxymethyl- cycloheptylmethyl)-benzamide |
| 18 | 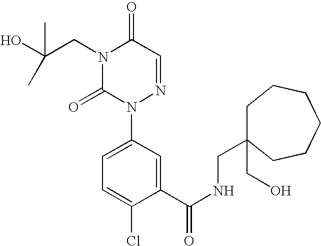 | 2-Chloro-N-(1-hydroxymethyl- cycloheptylmethyl)-5-[4-(2- hydroxy-2-methyl-propyl)-3,5- dioxo-4,5-dihydro-3H- [1,2,4]triazin-2-yl]-benzamide |
| 19 | 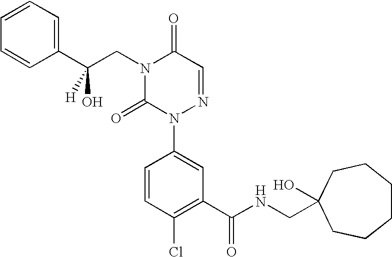 | 2-Chloro-N-(1-hydroxy- cycloheptylmethyl)-5-[4-(2- hydroxy-2-phenyl-ethyl)-3,5-dioxo- 4,5-dihydro-3h-[1,2,4]triazin-2-yl]- benzamide |
| 20 | 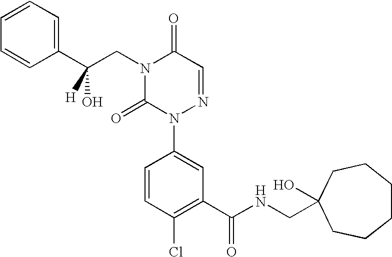 | 2-Chloro-N-(1-hydroxy- cycloheptylmethyl)-5-[4-(2- hydroxy-2-phenyl-ethyl)-3,5-dioxo- 4,5-dihydro-3H-[1,2,4]triazin-2-yl]- benzamide |
| 21 | 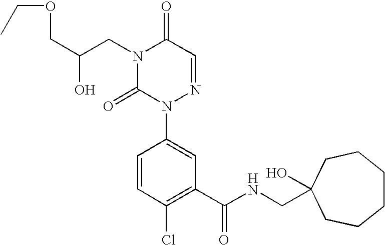 | 2-Chloro-5-[4-(3-ethoxy-2-hydroxy- propyl)-3,5-dioxo-4,5-dihydro-3H- [1,2,4]triazin-2-yl]-N-(1-hydroxy- cycloheptylmethyl)-benzamide |
| 22 | 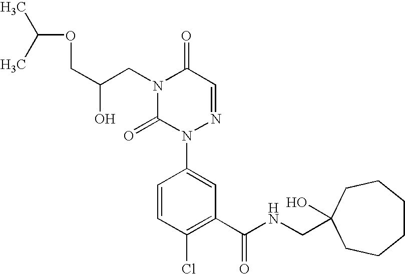 | 2-Chloro-N-(1-hydroxy- cycloheptylmethyl)-5-[4-(2- hydroxy-3-isopropoxy-propyl)-3,5- dioxo-4,5-dihydro-3H- [1,2,4]triazin-2-yl]-benzamide |
| 23 | 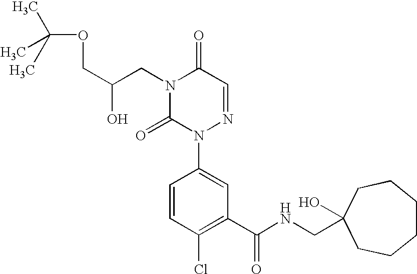 | 5-[4-(3-tert-Butoxy-2-hydroxy- propyl)-3,5-dioxo-4,5-dihydro-3H- [1,2,4]triazin-2-yl]-2-chloro-N-(1- hydroxy-cycloheptylmethyl)- benzamide |
| 24 | 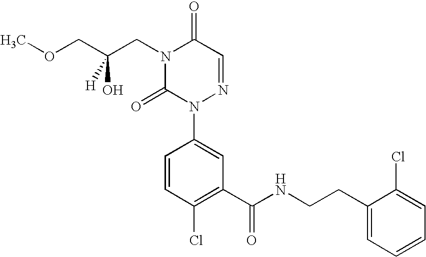 | 2-Chloro-N-[2-(2-chloro-phenyl)- ethyl]-5-[4-(2-hydroxy-3-methoxy- propyl)-3,5-dioxo-4,5-dihydro-3H- [1,2,4]triazin-2-yl]-benzamide |
| 25 | 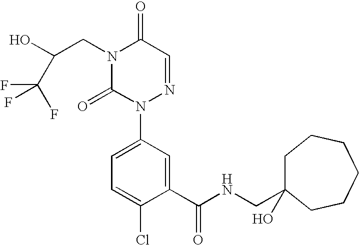 | 2-Chloro-5-[3,5-dioxo-4-(3,3,3- trifluoro-2-hydroxy-propyl)-4,5- dihydro-3H-[1,2,4]triazin-2-yl]-N- (1-hydroxy-cycloheptylmethyl)- benzamide |
| 26 | 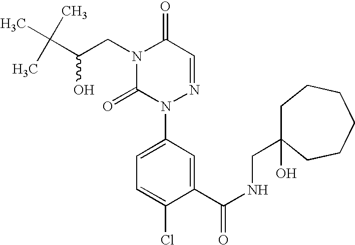 | 2-Chloro-N-(1-hydroxy- cycloheptylmethyl)-5-[4-(2- hydroxy-3,3-dimethyl-butyl)-3,5- dioxo-4,5-dihydro-3H- [1,2,4]triazin-2-yl]-benzamide |
| 27 | 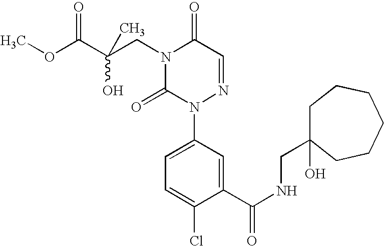 | 3-(2-{4-Chloro-3-[(1-hydroxy- cycloheptylmethyl)-carbamoyl]- phenyl}-3,5-dioxo-2,5-dihydro-3H- [1,2,4]triazin-4-yl)-2-hydroxy-2- methyl-propionic acid methyl ester |
| 28 | 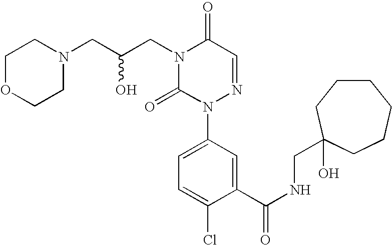 | 2-Chloro-N-(1-hydroxy- cycloheptylmethyl)-5-[4-(2- hydroxy-3-morpholin-4-yl-propyl)- 3,5-dioxo-4,5-dihydro-3H- [1,2,4]triazin-2-yl]-benzamide |
| 29 | 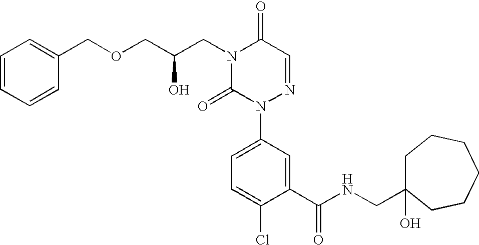 | 5-[4-(3-Benzyloxy-2-hydroxy- propyl)-3,5-dioxo-4,5-dihydro-3H- [1,2,4]triazin-2-yl]-2-chloro-N-(1- hydroxy-cycloheptylmethyl)- benzamide |
| 30 | 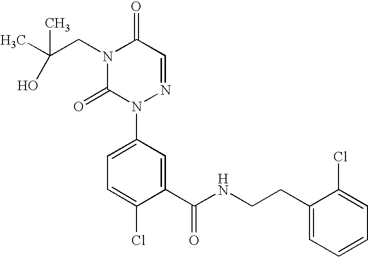 | 2-Chloro-N-[2-(2-chloro-phenyl)- ethyl]-5-[4-(2-hydroxy-2-methyl- propyl)-3,5-dioxo-4,5-dihydro-3H- [1,2,4]triazin-2-yl]-benzamide |
| 31 | 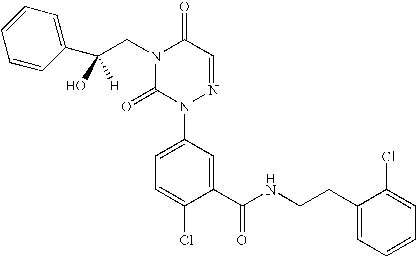 | 2-Chloro-N-[2-(2-chloro-phenyl)- ethyl]-5-[4-(2-hydroxy-2-phenyl- ethyl)-3,5-dioxo-4,5-dihydro-3H- [1,2,4]triazin-2-yl]-benzamide |
| 32 | 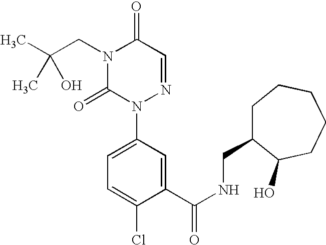 | 2-Chloro-N-(2-hydroxy- cycloheptylmetbyl)-5-[4-(2- hydroxy-2-methyl-propyl)-3,5- dioxo-4,5-dihydro-3H- [1,2,4]triazin-2-yl]-benzamide |
| 33 | 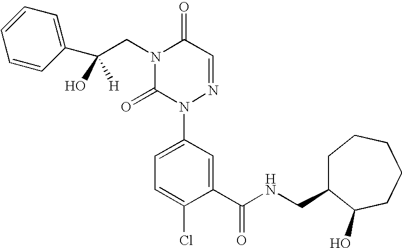 | 2-Chloro-N-(2-hydroxy- cycloheptylmethyl)-5-[4-(2- hydroxy-2-phenyl-ethyl)-3,5-dioxo- 4,5-dihydro-3H-[1,2,4]triazin-2-yl]- benzamide |
| 34 | 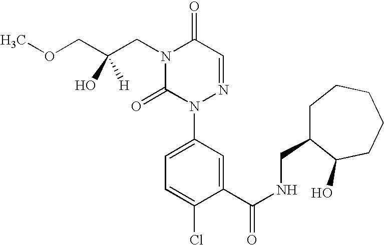 | 2-Chloro-N-(2-hydroxy- cycloheptylmethyl)-5-[4-(2- hydroxy-3-methoxy-propyl)-3,5- dioxo-4,5-dihydro-3H- [1,2,4]triazin-2-yl]-benzamide |
| 35 | 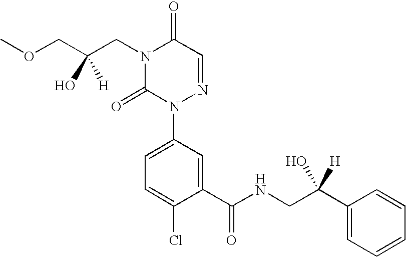 | 2-Chloro-5-[4-(2-hydroxy-3- methoxy-propyl)-3,5-dioxo-4,5- dihydro-3H-[1,2,4]triazin-2-yl]-N- (2-hydroxy-2-phenyl-ethyl)- benzamide |
| 36 | 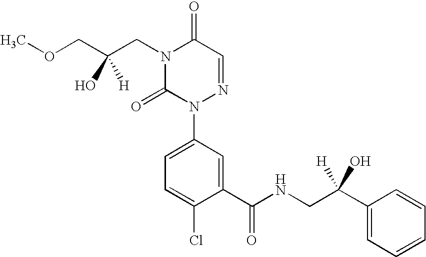 | 2-Chloro-5-[4-(2-hydroxy-3- methoxy-propyl)-3,5-dioxo-4,5- dihydro-3H-[1,2,4]triazin-2-yl]-N- (2-hydroxy-2-phenyl-ethyl)- benzamide |
| 37 | 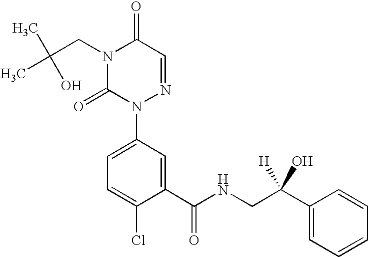 | 2-Chloro-5-[4-(2-hydroxy-2- methyl-propyl)-3,5-dioxo-4,5- dihydro-3H-[1,2,4]triazin-2-yl]-N- (2-hydroxy-2-phenyl-ethyl)- benzamide |
| 38 | 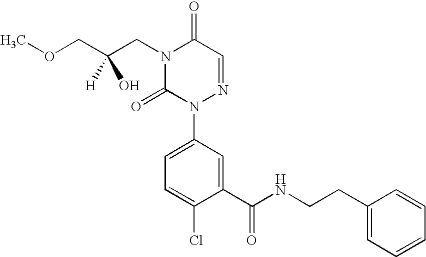 | 2-Chloro-5-[4-(2-hydroxy-3- methoxy-propyl)-3,5-dioxo-4,5- dihydro-3H-[1,2,4]triazin-2-yl]-N- phenethyl-benzamide |
| 39 | 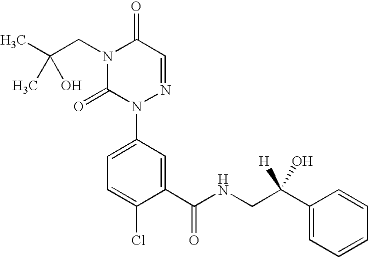 | 2-Chloro-5-[4-(2-hydroxy-2- methyl-propyl)-3,5-dioxo-4,5- dibydro-3H-[1,2,4]triazin-2-yl]-N- (2-hydroxy-2-phenyl-ethyl)- benzamide |
| 40 | 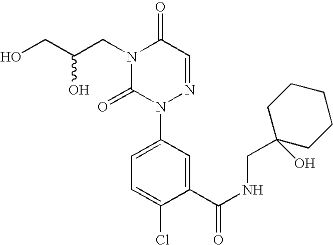 | 2-Chloro-5-[4-(2,3-dihydroxy- propyl)-3,5-dioxo-4,5-dihydro-3H- [1,2,4]triazin-2-yl]-N-(1-hydroxy- cyclohexylmethyl)-benzamide |
| 41 | 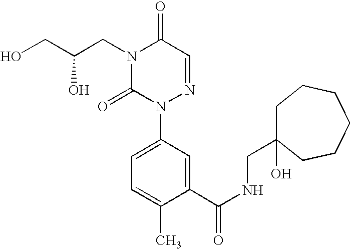 | 5-[4-(2,3-Dihydroxy-propyl)-3,5- dioxo-4,5-dihydro-3H- [1,2,4]triazin-2-yl]-N-(1-hydroxy- cycloheptylmethyl)-2-methyl- benzamide |
| 42 | 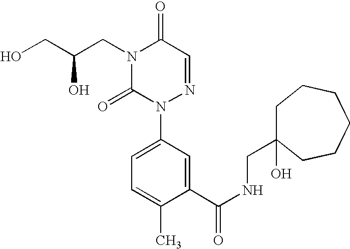 | 5-[4-(2,3-Dihydroxy-propyl)-3,5- dioxo-4,5-dihydro-3H- [1,2,4]triazin-2-yl]-N-(1-hydroxy- cycloheptylmethyl)-2-methyl- benzamide |

### III. Preparation of Crystalline 2-Chloro—N-(1-hydroxy-cycloheptylmethyl)-5-[4-(2R-hydroxy-3-methoxy-propyl)-3,5-dioxo-4,5-dihydro-3H-[1,2,4]triazin-2-yl]-5 benzamide

Various crystalline forms of a pharmaceutically active compound can be obtained by crystallizing the compound from one or more organic solvents. The resulting isolated crystalline form may contain one or more organic solvents —“residual organic solvent.” In certain cases, the amount of residual organic solvent in the crystalline form may need to be reduced to a level acceptable to a regulatory agency (See e.g., Dwivedi (November 2002) Pharmaceutical Tech. pages 42-46; and Guidance for Industry, Q3C Impurities: Residual organic solvents, U.S. Food and Drug Administration, Rockville, Md., December 1997), and that is compatible with charactistics such as stability, handling, etc., involved in producing the finished unit dosage form.

Example 61 of U.S. patent application Ser. No. 10/748,340, discloses a crystalline preparation of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide having a single melting endotherm at an onset of 105.8° C. as measured using differential scanning calorimetry (DSC), carried out at a heating rate of 5° C./min from 30-300° C. In certain embodiments of the present invention, crystalline preparations of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide of the present invention have a melting point onset of between 108° C.±0.5 and 112° C.±0.5 as measured by Differential Scanning Calorimetry.

In certain embodiments, a crystalline form of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide is produced by generating crystals of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide in solution comprising one or more organic solvents. Those of skill in art can vary factors including the concentration of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide in solution, solvent composition, the addition of a crystalline seed of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide, time and temperature at each step, the purity of the starting material of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide that is in solution, purity of the solvent(s) being employed, etc. to obtain the desired crystalline material.

In certain embodiments, the starting material is 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide that has been crystallized previously and then is dissolved into solution. In addition, techniques such as the heating and then cooling of a solution of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide can be employed to obtain crystalline material. In certain embodiments, the solution is cooled to 20-27° C., 10-22° C., 0-12° C., or −10 to 2° C., etc. In certain embodiments, the solution is heated to a temperature that is between 40-60° C. 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide may be added to a solution of one or more solvents and heated followed by one or more steps comprising concentrating the solution, cooling the solution to a lower temperature (e.g., 20-27° C., 10-22° C., 0-12° C., −10 to 2° C., etc.), and addition of a crystalline seed.

The crystallization of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide may be carried out at ambient, elevated or reduced temperatures and varying lengths of time depending on conditions such as the nature of the technique and the nature of the organic solvent(s) to achieve the desired results. Crystallization may be induced by steps that include changes in temperature (e.g., lowering the temperature), addition of solvents, and the addition of a small amount (e.g., a seed) of crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide to the crystallization solution.

2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide may be crystallized from a solution comprising a single organic solvent. 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide may be dissolved in a solvent such as acetone, acetonitrile, N,N-dimethylacetamide, N,N-dimethylformamide, dimethylsulfoxide, chloroform, ethyl acetate, and methanol by heating (e.g., to a temperature of between 50 and 70° C.) and then cooling the solution to a lower temperature until crystalline materials appears in solution.

In addition, 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide may be obtained from a solvent system comprising two or more solvents that are relatively miscible. Further, 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide may be crystallized from a first solution of one or more organic solvents in which the solvents are relatively miscible with each other and in which the compound is reasonably soluble and then combining it with one or more second solvents, that are relatively miscible with the first solution, in which 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide is much less soluble than in the first solution. Examples of solvents in which 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide is reasonably soluble include acetone, acetonitrile, N,N-dimethylacetamide, N,N-dimethylformamide, dimethylsulfoxide, dichloromethane, chloroform, ethanol, ethyl acetate, methylethyl ketone, methanol, and tetrahydrofuran. In certain embodiments, 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide is soluble at less than about 1 to about 20 mg/ml in the second solvent. In other embodiments, 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide is soluble at about 5 to about 20 mg/ml in the second solvent. In other embodiments, 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide is reasonably soluble in a solvent at about 50 to greater than 190 mg/ml. In certain embodiments, 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide is reasonably soluble in a solvent at about 20 to greater than 190 mg/ml. In other embodiments, 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide is reasonably soluble in a solvent at about 50 to about 180 mg/ml. Examples of suitable second solvents include cyclohexane, n-heptane, benzyl alcohol, hexane, isopropyl alcohol, diisopropyl ether, methylisobutyl ketone, toluene, and water. The second solvent may be combined with the first solution in a slow, medium, or rapid addition rate.

2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide can be dissolved in a solvent such as acetone by heating to a temperature such as 55° C. The acetone solution comprising 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide can then be cooled to ambient temperature and crystallization induced with the addition of a crystalline seed of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide, followed by the addition of n-heptane.

Crystalline 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide may be isolated from solution using one or more techniques, or combinations thereof, such as filtration, subjecting the sample to a stream of air or inert gas (e.g., nitrogen or argon), centrifugation, evaporation, and drying (e.g., under ambient conditions, or in a vacuum oven at ambient or elevated temperatures (e.g., 40-50° C.)). In certain embodiments, the crystalline material so prepared has less than 2.5% residual organic solvent. In certain embodiments, the crystalline material so prepared has composition has a melting point onset of between 108° C.±0.5 and 112° C.±0.5 as measured by Differential Scanning Calorimetry.

### Characterization Of Crystalline 2-Chloro-N-(1-Hydroxy-Cycloheptylmethyl)-5-[4-(2R-Hydroxy-3-Methoxy-Propyl)-3,5-Dioxo-4,5-Dihydro-3H-[1,2,4]Triazin-2-yl]-Benzamide

Crystalline material can be characterized using one more techniques including polarized light microscopy, differential scanning calorimetry, thermogravimetric analysis, and/or x-ray powder diffraction (“XPRD”) methods. For example, differential scanning calorimetry may be used to determine the thermal transitions of a sample with respect to temperature, including the melting point onset of the sample.

To perform an X-ray powder diffraction measurement on a XPRD instrument such as a Shimadzu or Bruker instrument, the sample is typically placed into a holder which has a cavity. The sample powder is pressed by a glass slide or equivalent to ensure a random surface and proper sample height. The sample holder is then placed into the instrument. The source of the X-ray beam is positioned over the sample, initially at a small angle relative to the plane of the holder, and moved through an arc that continuously increases the angle between the incident beam and the plane of the holder. Measurement differences associated with such X-ray powder analyses result from a variety of factors including: (a) errors in sample preparation (e.g., sample height), (b) instrument errors (e.g., flat sample errors), (c) calibration errors, (d) operator errors (including those errors present when determining the peak locations), and (e) preferred orientation. Calibration errors and sample height errors can result in a shift of all the peaks in the same direction and by the same amount. Small differences in sample height on a flat holder lead to large displacements in XRPD peak positions. A systematic study showed that, using a Shimadzu XRD-6000 in the typical Bragg-Brentano configuration, sample height differences of 1 mm led to peak shifts as high as 1°2θ (Chen, et al., J. Pharmaceutical and Biomedical Analysis, 2001; 26:63). These shifts can be identified from the X-ray diffractogram and can be corrected for by compensating for the shift (applying a systematic correction factor to all peak position values) or recalibrating the instrument.

Powder x-ray diffraction patterns may also be analyzed out on an Inel (capillary) diffractometer (e.g., an Inel XRG-3000 diffractometer, equipped with a Curved Position Sensitive (CPS) detector with a 20 range of 120 degrees). Real time data can be collected using CuK_{α} radiation starting at approximately 4°2θ at a resolution of 0.03° 2θ. The tube voltage and amperage can be set to values such as 40 kV and 30 mA, respectively. Samples are typically prepared for analysis by packing them into thin-walled glass capillaries. Each capillary is generally mounted onto a goniometer head that is motorized to permit spinning of the capillary during data acquisition. Instrument calibration can be performed daily using a silicon reference standard. The calculation of intensities from these diffractograms is within the skill of the art and involves using baseline subtraction to account for background scattering (e.g., scattering from the capillary).

As mentioned above, it is possible to rectify measurements from various XPRD instruments by applying a systematic correction factor can be applied to rectify measurements from various machines to bring the peak positions into agreement. In general, a correction factor will bring the peak positions into agreement with each other and will be in the range of 0 to 0.2°2θ.

In addition, crystalline material may be analyzed using ¹³C-solid state NMR techniques to determine the parts per million of one or more peaks in the ¹³C-solid-state spectrum. For example, crystalline material can be packed into a 4 mm ZrO spinner and the one-dimensional ¹³C spectra collected at ambient pressure using ¹H-¹³C cross-polarization magic angle spinning (CPMAS) at 293 K on a Bruker 4 mm BL CPMAS probe positioned into a wide-bore Bruker-Biospin Avance DSX 500 MHz NMR spectrometer. The sample can then be spun at 15.0 kHz with a cross-polarization contact time of 2.3 ms, with the decoupling power set to 85 kHz. The carbon spectra are typically referenced with an external sample of adamantane, setting its upfield resonance to a specific ppm value, e.g., 29.5 ppm.

The percentage of residual organic solvent (w/w) may be determined using techniques such as gas chromatography (“GC”) headspace analysis (see e.g., B'Hymer (2003) Pharm. Res. 20: 337-344). In GC headspace analysis, typically a sample of gas above the sample is collected and analyzed on a gas chromotography system that is coupled to a detection system such as a flame ionization detector (FID) or a mass spectrometer (MS). In addition, methods such as headspace solid-phase microextraction (SPME) may be used to determine residual organic solvent levels.

### IV. In Vitro Evaluation of Compounds

The activity of the compounds of the invention for the various disorders described above can be determined according to one or more of the following assays. All of the compounds of the invention that were tested had an IC₅₀ of less than 10 μM in the in vitro assay described below.

Preferably, the compounds of the invention have an IC₅₀ in the in vitro assays described below of less than 100 nM, more preferably less than 50 nM, and most preferably less than 10 nM. Still further, the compounds of the invention preferably have an IC₅₀ in the range of 0.01 nM-100 nM, more preferably between 0.05 nM-50 nM, and most preferably between 0.10 nM-10 nM.

### Pharmacological Analysis

Certain compounds such as benzoylbenzoyl adenosine triphosphate (bbATP) are known to be agonists of the P2X₇ receptor, effecting the formation of pores in the plasma membrane (Drug Development Research (1996), 37(3), p. 126). Consequently, when the receptor is activated using bbATP in the presence of ethidium bromide (a fluorescent DNA probe), an increase in the fluorescence of intracellular DNA-bound ethidium bromide is observed. Alternatively, the propidium dye YOPRO-1 can be substituted for ethidium bromide so as to detect uptake of the dye. The increase in fluorescence can be used as a measure of P2X₇ receptor activation and therefore to quantify the effect of a compound on the P2X₇ receptor.

In this manner, the compounds of the invention can be tested for antagonist activity at the P2X₇ receptor. 96-Well flat bottomed microtitre plates are filled with 250 μl of test solution comprising 200 μl of a suspension of THP-1 cells (2.5×10⁶ cells/ml, more preferably prestimulated as described in the literature with a combination of LPS and TNF to promote receptor expression) containing 10⁻⁴M ethidium bromide, 25 μl of a high potassium, low sodium buffer solution (10 mM Hepes, 150 mM KCl, 5 mM D-glucose and 1.0% FBS at pH 7.5) containing 10⁻⁵M bbATP, and 25 μl of the high potassium buffer solution containing 3×10⁻⁵M test compound (more preferably 5×10⁴M, more preferably 1×10⁴M, more preferably 1×10⁻³M). The plate is covered with a plastic sheet and incubated at 37° C. for one hour. The plate is then read in a Perkin-Elmer fluorescent plate reader, excitation 520 nm, emission 595 nm, slit widths: Ex 15 nm, Em 20 nm. For the purposes of comparison, bbATP (a P2X₇ receptor agonist) and pyridoxal 5-phosphate (a P2X₇ receptor antagonist) can be used separately in the test as controls. From the readings obtained, a pIC₅₀ figure can be calculated for each test compound, this figure being the negative logarithm of the concentration of test compound necessary to reduce the bbATP agonist activity by 50%.

In like manner, the compounds of the invention can be tested for antagonist activity at the P2X₇ receptor using the cytokine IL-1β as the readout. Blood collected from normal volunteers in the presence of heparin is fractionated using lymphocyte separation medium obtained from Organon Technica-(Westchester, Pa.). The region of the resulting gradient containing banded mononuclear cells is harvested, diluted with 10 ml of Maintenance Medium (RPMI 1640, 5% FBS, 25 mM Hepes, pH 7.2, 1% penicillin/streptomycin), and cells are collected by centrifugation. The resulting cell pellet was suspended in 10 ml of Maintenance Medium and a cell count was performed. In an average experiment, 2×10⁵ mononuclear cells are seeded into each well of 96-well plates in a total volume of 0.1 ml. Monocytes are allowed to adhere for 2 hours, after which the supernatants are discarded and the attached cells are rinsed twice and then incubated in Maintenance Medium overnight at 37° C. in a 5% CO₂ environment.

The cultured monocytes can be activated with 10 ng/ml LPS (E. coli serotype 055:B5; Sigma Chemicals, St. Louis, Mo.). Following a 2-hour incubation, the activation medium is removed, the cells are rinsed twice with 0.1 ml of Chase Medium (RPMI 1640, 1% FBS, 20 mM Hepes, 5 mM NaHCO₃, pH 6.9), and then 0.1 ml of Chase Medium containing a test agent is added and the plate is incubated for 30 minutes; each test agent concentration can be evaluated in triplicate wells. ATP then is introduced (from a 100 mM stock solution, pH 7) to achieve a final concentration of 2 mM and the plate is incubated at 37° C. for an additional 3 hours. Media were harvested and clarified by centrifugation, and their IL-1β content was determined by ELISA (R&D Systems; Minneapolis, Minn.).

### V. Pharmaceutically Acceptable Salts

The compounds of the present invention (e.g., compounds of Formula I) can be capable of further forming both pharmaceutically acceptable salts, including but not limited to acid addition and/or base salts. Pharmaceutically acceptable salts of the compounds of formula (I) include the acid addition and base salts (including disalts) thereof. Examples of suitable salts can be found for example in Stahl and Wermuth, Handbook of Pharmaceutical Salts: Properties, Selection, and Use, Wiley-VCH, Weinheim, Germany (2002); and Berge et al., “Pharmaceutical Salts,” J. of Pharmaceutical Science, 1977; 66:1-19.

Pharmaceutically acceptable acid addition salts of the compounds of Formula I include non-toxic salts derived from inorganic acids such as hydrochloric, nitric, phosphoric, sulfuric, hydrobromic, hydriodic, phosphorus, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include the acetate, aspartate, benzoate, besylate (benzenesulfonate), bicarbonate/carbonate, bisulfate, caprylate, camsylate (camphor sulfonate), chlorobenzoate, citrate, edisylate (1,2-ethane disulfonate), dihydrogenphosphate, dinitrobenzoate, esylate (ethane sulfonate), fumarate, gluceptate, gluconate, glucuronate, hibenzate, hydrochloride/chloride, hydrobromide/bromide, hydroiodide/iodide, isobutyrate, monohydrogen phosphate, isethionate, D-lactate, L-lactate, malate, maleate, malonate, mandelate, mesylate (methanesulfonate), metaphosphate, methylbenzoate, methylsulfate, 2-napsylate (2-naphthalene sulfonate), nicotinate, nitrate, orotate, oxalate, palmoate, phenylacetate, phosphate, phthalate, propionate, pyrophosphate, pyrosulfate, saccharate, sebacate, stearate, suberate, succinate sulfate, sulfite, D-tartrate, L-tartrate, tosylate (toluene sulfonate), and xinafoate salts, and the like of compounds of Formula I. Also contemplated are the salts of amino acids such as arginate, gluconate, galacturonate, and the like.

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metal hydroxides, or of organic amines. Examples of metals used as cations are aluminum, calcium, magnesium, potassium, sodium, and the like. Examples of suitable amines include arginine, choline, chloroprocaine, N,N′-dibenzylethylenediamine, diethylamine, diethanolamine, diolamine, ethylenediamine (ethane-1,2-diaamine), glycine, lysine, meglumine, N-methylglucamine, olamine, procaine (benzathine), and tromethamine.

The base addition salts of acidic compounds are prepared by contacting the free acid form with a sufficient amount of the desired base to produce the salt in the conventional manner. The free acid form may be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes of the present invention.

### VI. Pharmaceutical Compositions and Methods of Administration

This invention also provides for pharmaceutical compositions comprising a therapeutically effective amount of crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising less than 2.5% residual organic solvent admixed with at least one pharmaceutically acceptable carrier, and pharmaceutical compositions comprising: a therapeutically effective amount of a 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising residual organic solvent admixed with at least one pharmaceutically acceptable carrier, wherein said 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide has a melting point onset of between 108° C.±0.5 and 112° C.±0.5 as measured by Differential Scanning Calorimetry. Pharmaceutical compositions of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide may be prepared by admixing at least one pharmaceutically acceptable carrier with crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide.

The phrase “pharmaceutical composition” refers to a composition suitable for administration in medical or veterinary use. The phrase “therapeutically effective amount” means an amount of a compound, or a pharmaceutically acceptable salt thereof, sufficient to inhibit, halt, or allow an improvement in the disease being treated when administered alone or in conjunction with another pharmaceutical agent or treatment in a particular subject or subject population. For example in a human or other mammal, a therapeutically effective amount can be determined experimentally in a laboratory or clinical setting, or may be the amount required by the guidelines of the United States Food and Drug Administration, or equivalent foreign agency, for the particular disease and subject being treated.

It should be appreciated that determination of proper dosage forms, dosage amounts, and routes of administration is within the level of ordinary skill in the pharmaceutical and medical arts, and is described below.

A compound of the present invention can be formulated as a pharmaceutical composition in the form of a syrup, an elixir, a suspension, a powder, a granule, a tablet, a capsule, a lozenge, a troche, an aqueous solution, a cream, an ointment, a lotion, a gel, an emulsion, etc. Preferably, a compound of the present invention will cause a decrease in symptoms or a disease indicia associated with a IIL-1 mediated disorder as measured quantitatively or qualitatively.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired.

The powders and tablets contain from 1% to 95% (w/w) of the active compound. In certain embodiments, the active compound ranges from 5% to 70% (w/w). Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 1000 mg, preferably 1.0 mg to 100 mg, or from 1% to 95% (w/w) of a unit dose, according to the particular application and the potency of the active component. The composition can, if desired, also contain other compatible therapeutic agents.

Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington: The Science and Practice of Pharmacy, 20th ed., Gennaro et al. Eds., Lippincott Williams and Wilkins, 2000).

A compound of the present invention, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

The dose administered to a subject, in the context of the present invention should be sufficient to affect a beneficial therapeutic response in the subject over time. The term “subject” refers to a member of the class Mammalia. Examples of mammals include, without limitation, humans, primates, chimpanzees, rodents, mice, rats, rabbits, horses, livestock, dogs, cats, sheep, and cows.

The dose will be determined by the efficacy of the particular compound employed and the condition of the subject, as well as the body weight or surface area of the subject to be treated. The size of the dose also will be determined by the existence, nature, and extent of any adverse side-effects that accompany the administration of a particular compound in a particular subject. In determining the effective amount of the compound to be administered in the treatment or prophylaxis of the disorder being treated, the physician can evaluate factors such as the circulating plasma levels of the compound, compound toxicities, and/or the progression of the disease, etc. In general, the dose equivalent of a compound is from about 1 μg/kg to 100 mg/kg for a typical subject. Many different administration methods are known to those of skill in the art.

For administration, compounds of the present invention can be administered at a rate determined by factors that can include, but are not limited to, the pharmacokinetic profile of the compound, contraindicated drugs, and the side-effects of the compound at various concentrations, as applied to the mass and overall health of the subject. Administration can be accomplished via single or divided doses.

Examples of a typical tablet, parenteral, and patch formulation include the following:

TABLET FORMULATION EXAMPLE 1
Tablet Formulation

| Ingredient | Amount |
|---|---|
| 2-Chloro-N-(1-hydroxy- | 50 mg |
| cycloheptylmethyl)-5-[4-(2R- | |
| hydroxy-3-methoxy-propyl)-3,5- | |
| dioxo-4,5-dihydro-3H- | |
| [1,2,4]triazin-2-yl]-benzamide | |
| comprising 0.17% residual organic | |
| solvent | |
| Lactose | 80 mg |
| Cornstarch (for mix) | 10 mg |
| Cornstarch (for paste) | 8 mg |
| Magnesium Stearate (1%) | 2 mg |
| | 150 mg |

The compounds of the present invention (e.g., a compound of Formula I, or a pharmaceutically acceptable salt thereof) can be mixed with the lactose and cornstarch (for mix) and blended to uniformity to a powder. The cornstarch (for paste) is suspended in 6 mL of water and heated with stirring to form a paste. The paste is added to the mixed powder, and the mixture is granulated. The wet granules are passed through a No. 8 hard screen and dried at 50° C. The mixture is lubricated with 1% magnesium stearate and compressed into a tablet. The tablets are administered to a patient at the rate of 1 to 4 each day for treatment of a IL-1 mediated disease (e.g., rheumatoid arthritis).

### PARENTERAL SOLUTION FORMULATION EXAMPLE 1

In a solution of 700 mL of propylene glycol and 200 mL of water for injection can be added 20.0 g of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising 0.17% residual organic solvent. The mixture is stirred, and the pH is adjusted to 5.5 with hydrochloric acid. The volume is adjusted to 1000 mL with water for injection. The solution is sterilized, filled into 5.0 mL ampules, each containing 2.0 mL (40 mg of invention compound), and sealed under nitrogen. The solution is administered by injection to a subject suffering from a IL-1 mediated disease (e.g., rheumatoid arthritis) and in need of treatment.

### PATCH FORMULATION EXAMPLE 1

Ten milligrams of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising less than 0.17% residual organic solvent can be mixed with 1 mL of propylene glycol and 2 mg of acrylic-based polymer adhesive containing a resinous cross-linking agent. The mixture is applied to an impermeable backing (30 cm²) and applied to the upper back of a patient for sustained release treatment of a IL-1 mediated disease (e.g., rheumatoid arthritis).

### VII. Methods for Treating IL-1 Mediated Diseases

The compounds, compositions, and pharmaceutical compositions of the present invention can be administered to a subject suffering from a IL-1 mediated disease.

IL-1 mediated diseases can be treated prophylactically, acutely, and chronically using compounds of the present invention, depending on the nature of the disease. Typically, the host or subject in each of these methods is human, although other mammals can also benefit from the administration of a compound of the present invention.

In therapeutic applications, the compounds of the present invention can be prepared and administered in a wide variety of oral and parenteral dosage forms. The term “administering” refers to the method of contacting a compound with a subject. Thus, the compounds of the present invention can be administered by injection, that is, intravenously, intramuscularly, intracutaneously, subcutaneously, intraduodenally, parentally, or intraperitoneally. Also, the compounds described herein can be administered by inhalation, for example, intranasally. Additionally, the compounds of the present invention can be administered transdermally, topically, via implantation, transdermally, topically, and via implantation. In certain embodiments, the compounds of the present invention are delivered orally. The compounds can also be delivered rectally, bucally, intravaginally, ocularly, andially, or by insufflation.

The compounds utilized in the pharmaceutical method of the invention can be administered at the initial dosage of about 0.001 mg/kg to about 100 mg/kg daily. In certain embodiments, the daily dose range is from about 0.1 mg/kg to about 10 mg/kg. The dosages, however, may be varied depending upon the requirements of the subject, the severity of the condition being treated, and the compound being employed. Determination of the proper dosage for a particular situation is within the skill of the practitioner. Generally, treatment is initiated with smaller dosages, which are less than the optimum dose of the compound. Thereafter, the dosage is increased by small increments until the optimum effect under circumstances is reached. For convenience, the total daily dosage may be divided and administered in portions during the day, if desired. The term “treatment” includes the acute, chronic, or prophylactic diminishment or alleviation of at least one symptom or characteristic associated with or caused by the disorder being treated. For example, treatment can include diminishment of several symptoms of a disorder, inhibition of the pathological progression of a disorder, or complete eradication of a disorder. The compounds of the present invention can be co-administered to a subject. The term “co-administered” means the administration of two or more different pharmaceutical agents or treatments (e.g., radiation treatment) that are administered to a subject by combination in the same pharmaceutical composition or separate pharmaceutical compositions. Thus co-administration involves administration at the same time of a single pharmaceutical composition comprising two or more pharmaceutical agents or administration of two or more different compositions to the same subject at the same or different times. For example, a subject that is administered a first dosage that comprises a compound of the present invention at 8 a.m. and then is administered a second therapeutic agent at 1-12 hours later, e.g., 6 p.m., of that same day has been co-administered with a compound of the present invention and the second therapeutic agent. Alternatively, for example, a subject could be administered with a single dosage comprising a compound of the present invention and a second therapeutic agent at 8 a.m. has been co-administered with a compound of the present invention and the second therapeutic agent.

Thus, compounds of the invention can also be co-administered with compounds that are useful for the treatment of cancer (e.g., cytotoxic drugs such as TAXOL®, taxotere, GLEEVEC® (Imatinib Mesylate), adriamycin, daunomycin, cisplatin, etoposide, a vinca alkaloid, vinblastine, vincristine, methotrexate, or adriamycin, daunomycin, cis-platinum, etoposide, and alkaloids, such as vincristine, farnesyl transferase inhibitors, endostatin and angiostatin, VEGF inhibitors, and antimetabolites such as methotrexate. The compounds of the present invention may also be used in combination with a taxane derivative, a platinum coordination complex, a nucleoside analog, an anthracycline, a topoisomerase inhibitor, or an aromatase inhibitor). Radiation treatments can also be co-administered with a compound of the present invention for the treatment of cancers.

The compounds of the invention can also be co-administered with compounds that are useful for the treatment of a thrombolytic disease, heart disease, stroke, etc., (e.g., aspirin, streptokinase, tissue plasminogen activator, urokinase, anticoagulants, antiplatelet drugs (e.g., PLAVIX®; clopidogrel bisulfate), a statin (e.g., LIPITOR® (Atorvastatin calcium), ZOCOR® (Simvastatin), CRESTOR® (Rosuvastatin), etc.), a Beta blocker (e.g, Atenolol), NORVASC® (amlodipine besylate), and an ACE inhibitor (e.g., Accupril® (Quinapril Hydrochloride), Lisinopril, etc.).

The compounds of the invention can also be co-administered for the treatment of hypertension with compounds such as ACE inhibitors, lipid lowering agents such as statins, LIPITOR® (Atorvastatin calcium), calcium channel blockers such as NORVASC® (amlodipine besylate). The compounds of the present invention may also be used in combination with fibrates, beta-blockers, NEPI inhibitors, Angiotensin-2 receptor antagonists and platelet aggregation inhibitors.

For the treatment of inflammatory diseases, including rheumatoid arthritis, the compounds of the invention may be co-administered with agents such as TNF-α inhibitors such as anti-TNFα monoclonal antibodies (such as REMICADE®, CDP-870 and HUMIRA™ (adalimumab) and TNF receptor-immunoglobulin fusion molecules (such as ENBREL®), RITUXAN® (Rituximab), IL-1 inhibitors, receptor antagonists or soluble IL-1Rα (e.g. KINERET™ or ICE inhibitors), an IL-6 monoclonal antibody, an IL-6 receptor monoclonal antibody (e.g., MRA (Chugai)), an M-CSF monoclonal antibody, nonsteroidal anti-inflammatory agents (NSAIDS), piroxicam, diclofenac, naproxen, flurbiprofen, fenoprofen, ketoprofen ibuprofen, fenamates, mefenamic acid, indomethacin, sulindac, apazone, pyrazolones, phenylbutazone, aspirin, COX-2 inhibitors (such as CELEBREX® (celecoxib), VIOXX®) (rofecoxib), BEXTRA® (valdecoxib) and etoricoxib, metalloprotease inhibitors (preferably MMP-13 selective inhibitors), NEUROTIN®, pregabalin, low dose methotrexate, sulfasalazine, leflunomide, hydroxychloroquine, d-penicillamine, auranofin or parenteral or oral gold.

The compounds of the invention may be co-administered with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, rofecoxib and etoricoxib, analgesics and intraarticular therapies such as corticosteroids and hyaluronic acids such as hyalgan and synvisc.

The compounds of the invention may also be co-administered with antiviral agents such as Viracept, AZT, aciclovir and famciclovir, and antisepsis compounds such as Valant.

The compounds of the present invention may further be co-administered with CNS agents such as antidepressants (such as sertraline), anti-Parkinsonian drugs (such as deprenyl, L-Dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), NEURONTIN®, pregabalin, and anti-Alzheimer's drugs such as ARICEPT®, tacrine, propentofylline or metrifonate.

The compounds of the present invention may additionally be co-administered with osteoporosis agents such as EVISTA® (raloxifene hydrochloride), droloxifene, lasofoxifene or FOSAMAX® and immunosuppressant agents such as FK-506 and rapamycin.

### EXAMPLES

It is understood that the examples and embodiments described herein are for illustrative purposes only and that various modifications or changes in light thereof will be suggested to persons skilled in the art and are to be included within the spirit and purview of this application and the scope of the appended claims. All publications, patents, and patent applications cited herein are hereby incorporated by reference in their entirety for all purposes.

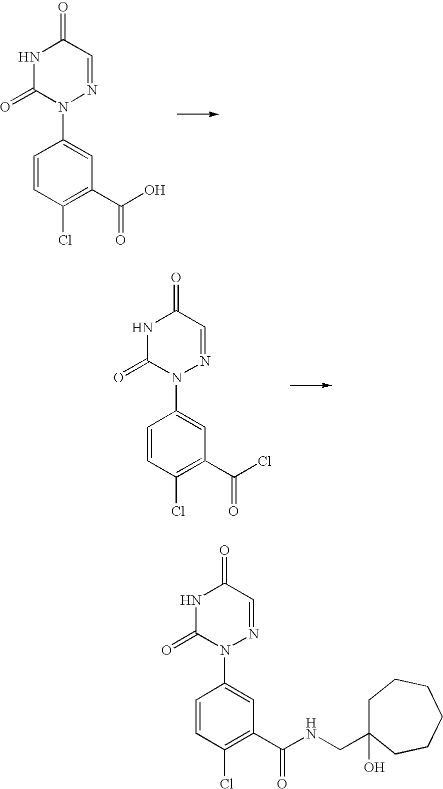

### Intermediate 1. 2-Chloro-5-(3,5-dioxo-4,5-dihydro-3H-[1,2,4]triazin-2-yl)-N-(1-hydroxy-cycloheptylmethyl)-benzamide

To a 200 L glass lined reactor, 7.50 kg of 2-Chloro-5-(3,5-dioxo-4,5-dihydro-3H-[1,2,4]triazin-2-yl)-benzoic acid and 72 L of dichloromethane was added and allowed to stir at 20-22° C. To this suspension was added, 3.75 L of oxalyl chloride over five minutes followed by 75 ml of N,N-Dimethylformamide (DMF). The resulting acid chloride (2-Chloro-5-(3,5-dioxo-4,5-dihydro-3H-[1,2,4]triazin-2-yl)-benzoyl chloride) was then isolated on a filter, washed with isopropyl ether, and blown dry with nitrogen gas. While the acid chloride was being dried on the filter 4.03 kg of 1-aminomethyl-cycloheptanol HCl, 2.31 kg of sodium hydroxide, and 52.5 L of water was added to the 200 L reactor and allowed to stir until a homogeneous solution was formed. The acid chloride was then added to this solution and stirred for 4 h at 20-22° C. at which time an HPLC assay showed reaction completion. The reaction mixture was then adjusted to pH 3 with 6N HCl followed by isolation of the crude Intermediate 1 (2-Chloro-5-(3,5-dioxo-4,5-dihydro-3H-[1,2,4]triazin-2-yl)-N-(1-hydroxy-cycloheptylmethyl)-benzamide) on a filter. The reactor and filter were washed with water and the crude Intermediate 1 was blown dry. Intermediate 1 was then recrystalized by taking the material on the filter up in 75 L of methanol, heating to 70° C., adding 37.5 L water, and cooling to 20° C. After stirring for 12 h, Intermediate 1 was isolated on a filter and washed with a water:methanol (2:1) solution and allowed to dry for 48 h in a vacuum dryer. 8.0 kg of Intermediate 1 was isolated (73% yield). ¹H NMR spectroscopy was performed on a Varian 400 MHz spectrometer with d₆-DMSO as solvent. ¹H NMR δ 12.37 (s, 1H), 8.32-8.34 (t, 1H), 7.65 (s, 1H), 7.53-7.59 (m, 3H) 4.24 (s, 1H), 3.32 (s, 1H), 2.49-3.20 (d, 1H), 2.48 (m, 2H), 1.33-1.63 (m, 10H). MS: 393.1 (M+1).

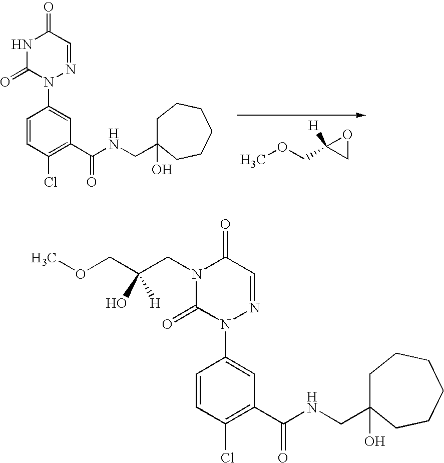

### Example 1

### 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide

To a 200 L glass lined reactor, DMF 9.6 L and Silica gel 4.12 kg was added and allowed to stir for a half an hour, the resulting mixture remained as a slurry. Intermediate 1 (2.75 kg, 7 moles) was then added and at this point the mixture became a solution after stirring for half an hour. R-(−)-methyl glycidyl ether (954 g, 10.5 moles) was then added drop wise while the temperature of the reactor was heated at 80° C. for 16 h. After HPLC assay showed reaction completion the reactor was cooled to 25° C. and ethyl acetate 82.5 L was added to precipitate the silica gel. The silica gel was then filtered off and the ethyl acetate layer was extracted three successive times with 14 L of saturated sodium bicarbonate, and then once with 14 L of water. The ethyl acetate layer was atmospherically concentrated at 76-78° C. to about 14 L and then seeded with crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide to initiate crystallization. 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide was isolated on a filter and dried in vacuum oven over the weekend to give 2.5 kg, 74% yield. ¹H & ¹³C NMR spectra, Mass spec. of 481.4 (M+1), & HPLC-UV were consistent with the structure of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide.

### Example 2

Intermediate 1 (1 g, 2.5 mmol), DMF (3 ml, 3 vol), (R)-(−)-Glycidyl methyl ether (230 mg, 2.6 mmol, 1.05 equiv.), and boron trifluoride diethyl etherate (3.6 mg, 0.025 mmol, 0.1 mol %) were combined in a test tube and heated under nitrogen to 80° C. for 16 hours. BPLC-UV analysis on a ZORBAX® SB-CN column (0.2% phosphoric acid in water:acetonitrile (1:1)) showed roughly a 9:1 ratio of Intermediate 1 to the product 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide.

### Example 3

To a 200 L glass lined reactor 4.3 kg of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide was synthesized as in Example 1 was added followed by the addition of ethyl acetate (130 L). The reactor was then heated to 78° C. taking 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide into solution. The reactor was then cooled to 25° C. and extracted with water (21.5 L). The ethyl acetate layer was then transferred to a 55 gal drum. Through an inline filter the ethyl acetate layer was transferred back to the 200 L reactor and atmospherically concentrated to about 20 L.

An aliquot was taken and placed in a round bottom flask, and crystallization was initiated by scratching with a metal rod. The mixture was filtered and dried under vacuum to give the seed crystal material. The seed crystal material was added back to the reactor, and after several hours of stirring a thick slurry was observed at 0° C. n-Hexane (8.6 L) was then added in one portion via vacuum to the reactor and the reactor was allowed to stir at −10° C. overnight. The material was isolated on a filter and dried over the weekend in a vacuum oven. GC-headspace analysis indicated the material contained 2.6% residual ethyl acetate. The material had a melting point onset of 106.5° C. as determined using differential scanning calorimetry (see below).

GC-headspace was determined in these examples with an Agilent GC 6850 Model G2630A, Hewlett Packard Headspace Auto sampler 7694 and a DB642, DB-624, 30 meters×0.32 mm I.D. fused silica, 1.8 μm film column. The method used had an Oven Temperature Program of 400 (5 minutes), followed by heating at 2° C./minute to 46° C., followed by heating at 25° C./minute to 225° C., and holding for 2 minutes at 225° C. The injector was at 180° C. The column flow was nitrogen gas at a flow of approximately 8.5 psi or 1.6 ml/minute. The split flow was approximately 47 ml/minute. The split ratio was approximately 30:1. An FID detector at 250° C. was employed.

### Example 4

To a 50 L glass lined reactor 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide (3.9 kg) (from Example 3) and acetone (11.7 L) was added. The reactor was then heated to 55° C. taking 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide into solution. The reactor was then cooled over 4 h to 20° C. crystallization was induced by adding 2 g of seed crystal of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide to the reactor. After stirring overnight at 20° C. a thick slurry was observed. A sample was filtered and then vacuum dried. GC headspace analysis gave 0.21% residual acetone (Example 4, Sample A).

n-Heptane (24 L) was then added to a head tank via an inline filter and added over 4 h to the 50 L reactor. The mixture was allowed to crystallize overnight at 200° C. A sample was filtered and then vacuum dried. GC headspace analysis gave 0.18% residual acetone (Example 4, Sample B). The material was isolated on a filter and blown dry with nitrogen gas overnight followed by drying in a vacuum oven (40-50° C.) over the weekend. GC headspace analysis gave 0.17% residual acetone (Example 4, Sample C).

The material was then dried in a vacuum oven (40-50° C.) for about an additional 5.5 hours to give Example 4, Sample D.

### Differential Scanning Calorimetry of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide

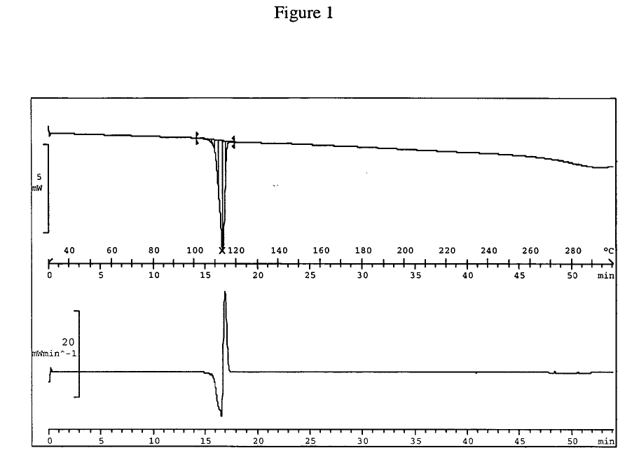

Differential Scanning Calorimetry (DSC) was carried out on a Mettler-Toledo 822e Differential Scanning Calorimeter on Example 4, Sample A; Example 4, Sample B; Example 4, Sample C; and Example 4, Sample D. The method employed 1-5 mg of sample aluminum 40 μl pan, with a start temperature of 30° C. and a final temperature of 300° C. The heating rate was 5° C./minute. The sampling interval was 1 second. The nitrogen gas flow rate was 60 ml/min. The DSC thermal profile of Example 4, Sample D is shown in FIG. 1. A melting onset temperature was detected at about 110.5° C. The melting onset temperatures for Example 4, Sample A; Example 4, Sample B; and Example 4, Sample C are reported in Table 2.

TABLE 2

| Sample | DSC mp |
|---|---|
| Example 4, Sample A | 111.3° C. |
| Example 4, Sample B | 111.4° C. |
| Example 4, Sample C | 111.3° C. |
| Example 4, Sample D | 110.5° C. |

### Solid-State ¹³C-NMR of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide, Example 4, Sample D

Approximately 70 mg of Example 4, Sample D was tightly packed into a 4 mm ZrO spinner for the sample analyzed. The one-dimensional ¹³C spectra were collected at ambient pressure using ¹H-¹³C cross-polarization magic angle spinning (CPMAS) at 293 K on a Bruker 4 mm BL CPMAS probe positioned into a wide-bore Bruker-Biospin Avance DSX 500 MHz NMR spectrometer. The sample was spun at 15.0 kHz corresponding to the maximum specified spinning speed for the 4 mm spinners. The fast spinning speed minimized the intensities of the spinning side bands. To optimize the signal sensitivity, the cross-polarization contact time was adjusted to 2.3 ms, and the decoupling power was set to 85 kHz. The carbon spetra were acquired with 5,940 scans with a recycle delay of 10 second. They were referenced using an external sample of adamantane, setting its upfield resonance to 29.5 ppm.

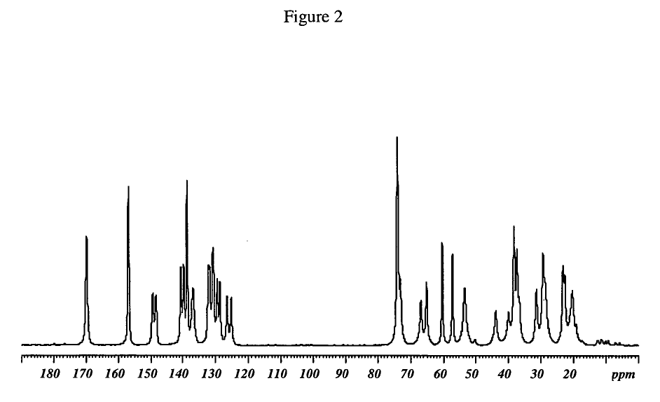

The resulting 1³C CPMAS spectrum of Example 4, Sample D material is shown in FIG. 2. The carbon peak list is given in Table 3. Please note that the numbering of the carbon peaks in Table 3 does not correspond to the numbering of carbon atoms in the 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]benzamide molecule (Structure 1). Some of the carbons in the molecule showed two ¹³C peaks which agrees with different conformation per asymmetric unit observed in the single crystal structure.

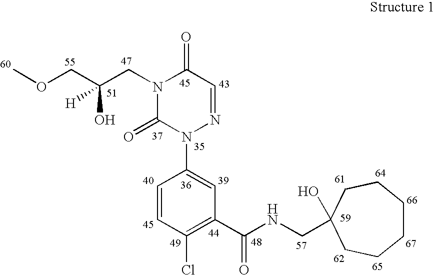

TABLE 3

| | | Intensity | | | Intensity |
|---|---|---|---|---|---|
| | ¹³C shift | (arbitrary | | ¹³C shift | (arbitrary |
| Peak | (ppm) | units) | Peak | (ppm) | units) |
| 1 | 169.8 | 6.22 | 18 | 66.9 | 2.42 |
| 2 | 156.8 | 9.11 | 19 | 65.1 | 3.55 |
| 3 | 149.3 | 2.83 | 20 | 60.4 | 5.82 |
| 4 | 148.4 | 2.77 | 21 | 57.2 | 5.19 |
| 5 | 140.6 | 4.38 | 22 | 53.4 | 3.14 |
| 6 | 139.7 | 4.56 | 23 | 50.4 | 0.13 |
| 7 | 138.7 | 9.47 | 24 | 43.9 | 1.85 |
| 8 | 136.9 | 3.13 | 25 | 40.0 | 1.76 |
| 9 | 132.2 | 4.5 | 26 | 38.2 | 6.84 |
| 10 | 131.9 | 4.49 | 27 | 37.3 | 5.47 |
| 11 | 130.8 | 5.5 | 28 | 36.6 | 2.7 |
| 12 | 129.5 | 3.7 | 29 | 31.4 | 3.11 |
| 13 | 128.7 | 3.55 | 30 | 29.3 | 5.2 |
| 14 | 126.5 | 2.71 | 31 | 23.2 | 4.48 |
| 15 | 125.2 | 2.6 | 32 | 22.7 | 3.96 |
| 16 | 74.0 | 12 | 33 | 20.4 | 3.04 |
| 17 | 73.1 | 3.68 | 34 | 19.2 | 1.06 |

### Powder X-Ray Diffraction (PXRD) analysis of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide (Example 4, Sample D)

The X-ray powder diffraction pattern was collected using a Bruker D5000 diffractometer equipped with CuK_{α} (fine focus x-ray tube) radiation (Generator:40 kV, 30 mA), fixed slits (Divergence slit: 1 mm; Scattering slit: 1 mm; Receiving slit: 0.6 mm), and a Kevex PSI or Sol-X solid state detector. Data was collected from 3.0 to 40.0 degrees in two theta using a step size of 0.04 degree and a step time of 1.0 second. The scan mode was continuous. The 2-theta values are reported in table 4.

TABLE 4
peak list (2-theta ± 0.2)
(Peak intensity may vary depending on the particle size and morphology)

| Angle 2- | | Angle 2- | |
|---|---|---|---|
| Theta ^{∘} | Intensity % | Theta ^{∘} | Intensity % |
| 7.8 | 11 | 23.3 | 13.1 |
| 8.1 | 100 | 23.9 | 9.8 |
| 10.5 | 10.3 | 24.3 | 14.5 |
| 11.7 | 21.3 | 24.6 | 7.6 |
| 13.2 | 5.2 | 25.1 | 9.9 |
| 13.7 | 8.1 | 25.9 | 9.2 |
| 14.3 | 5.7 | 26.2 | 12.7 |
| 14.9 | 21.6 | 27.1 | 30.9 |
| 15.6 | 5.2 | 27.6 | 9.6 |
| 16.4 | 86.9 | 28.2 | 5.2 |
| 17.3 | 17.5 | 28.7 | 4.9 |
| 17.7 | 17.3 | 28.8 | 4.9 |
| 18.3 | 19 | 29.4 | 6.4 |
| 18.9 | 9.2 | 30.0 | 10.2 |
| 19.1 | 9.9 | 30.3 | 14 |
| 19.7 | 85.9 | 30.9 | 7.9 |
| 20.3 | 9.5 | 31.1 | 7.7 |
| 20.9 | 8.8 | 31.9 | 6.2 |
| 21.2 | 33.3 | 33.4 | 5.9 |
| 21.6 | 24 | 33.8 | 6.4 |
| 22.2 | 33.8 | 34.3 | 7.5 |
| 22.6 | 17.6 | 35.2 | 8.9 |
| 22.8 | 6.7 | 37.1 | 6.2 |

The x-ray powder diffraction patterns of Example 4, Sample A; Example 4, Sample B; and Example 4, Sample C were consistent with that of Example 4, Sample D.

### Examples 5-13

The starting material for Examples 5-13 was from Example 3. The x-ray powder diffraction patterns of Examples 5-13 were consistent with that of Example 4, Sample D.

### Example 5

5 g of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide was taken up in 50 ml of n-Butyl Acetate at 125° C. then concentrated to 25 ml the resulting solids were filtered and dried in a vacuum oven.

### Example 6

5 g of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide was taken up in 25 ml of methyl ethyl ketone (MEK) at 70° C. 25 ml of Hexanes was added and the resulting solids were filtered off and dried in a vacuum oven.

### Example 7

19 g of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide was passed through a hand sieve and dried in vacuum oven.

### Example 8

15.5 g of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide was taken up in 75 ml of methanol at 50° C. the solution was then cooled to ambient temperature and 75 ml of water was added forming an oil that after 24 hours turned to solids. The solids were filtered off and dried in a vacuum oven.

### Example 9

1 g of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide was heated to 60° C. in 10 ml of n-Heptane for 4 hours. The mixture was then cooled to ambient temperature and the solids were filtered and dried in a vacuum oven.

### Example 10

1 g of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide was heated to 60° C. in 10 ml of Diisopropyl Ether for 4 hours. The mixture was then cooled to ambient temperature and the solids were filtered and dried in a vacuum oven.

### Example 11

10 g of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide was heated to 60° C. in 100 ml of Diisopropyl Ether for 4 hours. The mixture was then cooled to ambient temperature and the solids were filtered and dried in a vacuum oven.

### Example 12

10 g of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide was heated to 60° C. in 1000 ml of n-Heptane for 4 hours. The mixture was then cooled to ambient temperature and the solids were filtered and dried in a vacuum oven.

### Example 13

10 g of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide was taken up in 30 ml of Acetone at 50° C. The mixture was cooled to ambient temperature and 60 ml of n-Heptane was added. The solids were filtered off and dried in a vacuum oven.

### Example 15

50 mg of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide was dissolved in 1 mL of acetone, tetrahydrofuran or methyl ethyl ketone. The solution was heated briefly to ensure dissolution. To the solution, 2.5 mL of diisopropyl ether was added at 25° C. to afford crystalline solids having x-ray diffraction patterns consistent with that of Example 4, Sample D.

### Example 16

50 mg of 2-chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide was dissolved 1 mL of acetone, tetrahydrofuran or methyl ethyl ketone. The solution was heated briefly to ensure dissolution. The solution was added to 2.5 mL of diisopropyl ether at 25° C. to afford crystalline solids having x-ray diffraction patterns consistent with that of Example 4, Sample D.

### Example 17

### 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide

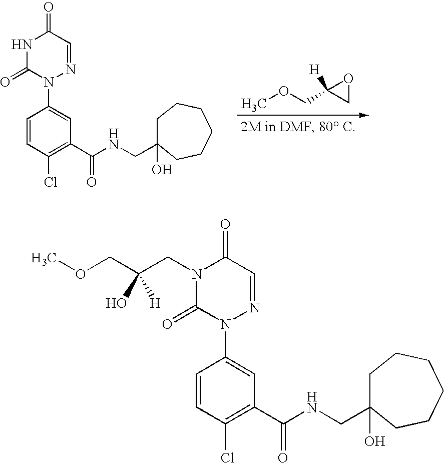

2.25 kg (1.00 equivalent) 2-Chloro-5-(3,5-dioxo-4,5-dihydro-3H-[1,2,4]triazin-2-yl)-N-(1-hydroxy-cycloheptyl-methyl)-benzamide, 0.780 kg (1.5 equivalents) R-methyl glycidyl ether and 3.38 kg silica gel (JT Baker Lot No. X46590) were combined in 6.75 L of N,N-dimethylformamide and heated to 80° C. The reaction mixture was cooled and 76.5 L (68.1 kg) ethyl acetate was added to precipitate out the silica gel, which was then filtered off. The ethyl acetate phase was then washed with saturated sodium bicarbonate (4.50 kg/2.9 L) to remove residual 2-Chloro-5-(3,5-dioxo-4,5-dihydro-3H-[1,2,4]triazin-2-yl)-N-(1-hydroxy-cycloheptyl-methyl)-benzamide, followed by concentration of the ethyl acetate layer to a lower volume. 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide was crystallized out of the ethyl acetate phase and isolated by filtration.

A sample of the resulting 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide was isolated and dried. GC headspace analysis for residual solvents, as described in Example 4, gave identified the following residual solvent concentrations.

| Methanol | Ethanol | Acetone | Ethyl Acetate | n-Heptane | 2-methoxymethyl-oxirane |
|---|---|---|---|---|---|
| — | — | 0.2% | <0.01% | 0.01% | <0.01%-ND |

ND is not detected

1. A method of preparing a compound of formula I 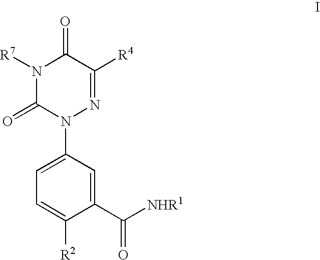
or a pharmaceutically acceptable salt thereof,
wherein R¹ is (C₁-C₆)alkyl, optionally substituted by (C₃-C₈)cycloalkyl, phenyl, naphthyl, 5 or 6-membered heterocycloalkyl, or a 5- or 6-membered heteroaryl, wherein each of said (C₁-C₆)alkyl, (C₃-C₈)cycloalkyl, phenyl, naphthyl, 5 or 6-membered heterocycloalkyl, or 5- or 6-membered heteroaryl are optionally substituted by one to three moieties independently selected from the group consisting of hydroxy, halo, CN—, (C₁-C₆)alkyl, HO(C₁-C₆)alkyl, (C₁-C₆)alkyl-NH(C═O)—, NH₂(C═O)—, (C₁-C₆)alkoxy, or (C₃-C₈)cycloalkyl;
R² is hydrogen, halo, —CN, or (C₁-C₆)alkyl, wherein said (C₁-C₆)alkyl is optionally substituted by one to three moieties, independently selected from the group consisting of halo, hydroxy, amino, —CN, (C₁-C₆)alkyl, (C₁-C₆)alkoxy, —CF₃, CF₃O—, (C₁-C₆)alkyl-NH—, [(C₁-C₆)alkyl]₂—N—, (C₁-C₆)alkyl-S—, (C₁-C₆)alkyl-(S═O)—, (C₁-C₆)alkyl-(SO₂)—, (C₁-C₆)alkyl-O—(C═O)—, formyl, (C₁-C₆)alkyl-(C═O)—, and (C₃-C₆)cycloalkyl;
wherein R⁴ is independently selected from the group consisting of hydrogen, halo, hydroxy, —CN, HO—(C₁-C₆)alkyl, (C₁-C₆)alkyl optionally substituted with one to three fluoro, (C₁-C₆)alkoxy optionally substituted with one to three fluoro, HO₂C—, (C₁-C₆)alkyl-O—(C═O)—, R⁵R⁶N(O₂S)—, (C₁-C₆)alkyl-(O₂S)—NH—, (C₁-C₆)alkyl-O₂S—[(C₁-C₆)alkyl-N]—, R⁵R⁶N(C═O)—, R⁵R⁶N(CH₂)ₘ—, phenyl, naphthyl, (C₃-C₈)cycloalkyl, 5- or 6-membered heteroaryl, 5 or 6-membered heterocycloalkyl, phenyl-O—, naphthyl-O—, (C₃-C₈)cycloalkyl-O—, 5- or 6-membered heteroaryloxy and 5 or 6-membered heterocycloalkyl-O—; and
R⁷ is —CH₂—C(R¹⁰R¹¹)—OH, wherein R¹⁰ and R¹¹ are independently selected from the group consisting of:
hydrogen, phenyl, and (C₁-C₆)alkyl optionally substituted with one to three halos, hydroxy, —CN, (C₁-C₆)alkoxy-, ((C₁-C₆)alkyl)ₙ—N—, (C₁-C₆)alkyl-(C═O)—, (C₃-C₈)cycloalkyl-(C═O)—, 5 or 6-membered heterocycloalkyl-(C═O)—, phenyl-(C═O)—, naphthyl-(C═O)—, 5- or 6-membered heteroaryl-(C═O)—, (C₁-C₆)alkyl-(C═O)O—, (C₁-C₆)alkyl-O(C═O)—, (C₃-C₈)cycloalkyl, phenyl, naphthyl, 5 or 6-membered heterocycloalkyl, and 5- or 6-membered heteroaryl;
R⁵ and R⁶ are each independently selected from the group consisting of hydrogen, (C₁-C₆)alkyl, HO—(C₂-C₆)alkyl and (C₃-C₈)cycloalkyl, or R⁵ and R⁶ may optionally be taken together with the nitrogen atom to which they are attached to form a 5 or 6-membered heterocycloalkyl;
n is one or two; and
m is one or two;
wherein said method comprises reacting a compound of formula II 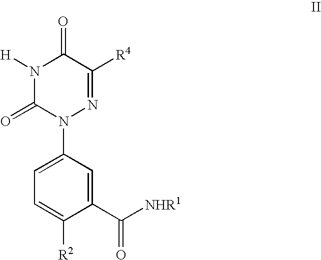
with a compound of Formula VII 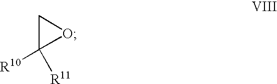
in the presence of at least one Lewis acid.

2. The method of claim 1 wherein said Lewis acid is selected from
(a) boron trifluoride diethyl etherate;
(b) Al₂O₃, Ti(O—Prⁱ)₄, LiClO₄, or Zn(OAc)₂;
(c) Eu(OTf)₃, Dy(OTf)₃, Ho(OTf)₃, Er(OTf)₃, Lu(OTf)₃, Yb(OTf)₃, Nd(OTf)₃, Gd(OTf)₃, Lu(OTf)₃, La(OTf)₃, Pr(OTf)₃, Tm(OTf)₃, Sc(OTf)₃, Sm(OTf)₃, AgOTf, or Y(OTf)₃;
(d) AlCl₃, AlI₃, AlF₃, AlBr₃, AsCl₃, AsI₃, AsF₃, AsBr₃, BCl₃, BBr₃, BI₃, BF₃, FeCl₃, FeBr₃, FeI₃, FeF₃, FeCl₂, FeBr₂, FeI₂, FeF₂, GaCl₃, GaI₃, GaF₃, GaBr₃, MgCl₂, MgI₂, MgF₂, MgBr₂, NbCl₅, SbCl₃, SbI₃, SbF₃, SbBr₃, SbCl₅, SbI₅, SbF₅, SbBr₅, SnCl₂, SnI₂, SnF₂, SnBr₂, SnCl₄, SnI₄, SnF₄, SnBr₄, TiBr₄, TiCl₂, TiCl₃, TiCl₄, TiF₃, TiF₄, TiI₄, ZnCl₂, ZnI₂, ZnF₂, or ZnBr₂.
(e) BF₃BCl₃-SMe₂, BI₃-SMe₂, BF₃—SMe₂, BBr₃-SMe₂, BF₃.OEt₂, Et₂AlCl, EtAlCl₂, MgCl₂-OEt₂, MgI₂-OEt₂, MgF₂—OEt₂, MgBr₂-OEt₂, Et₂AlCl, EtAlCl₂, or Zn(OAc)₂; and
(f) (CH₃CO₂)₂Co, CoBr₂, CoCl₂, CoF₂, CoI₂, Co(NO₃)₂, cobalt (II) triflate, cobalt (II) tosylate, (CH₃CO₂)₂Cu, CuBr₂, CuCl₂, CuF₂, CuI₂, Cu(NO₃)₂, copper (II) triflate, copper (II) tosylate, (CH₃CO₂)₂Ni, NiBr₂, NiCl₂, NiF₂, NiI₂, Ni(NO₃)₂, nickel (II) triflate, or nickel (II) tosylate.

3. The method of claim 1 wherein said Lewis acid is a silica gel.

4. The method of claim 3 wherein said compound of formula VIII is (R)-(−)-glycidyl methyl ether.

5. The method of claim 3 wherein said compound of formula II is 2-Chloro-5-(3,5-dioxo-4,5-dihydro-3H-[1,2,4]triazin-2-yl)-N-(1-hydroxy-cycloheptylmethyl)-benzamide.

6. The method of claim 3 wherein said compound of formula I is 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide.

7. The method of claim 1 wherein R¹ is a (C₁-C₄)alkyl, optionally substituted by (C₃-C₈)cycloalkyl; wherein said (C₁-C₄)alkyl or (C₃-C₈)cycloalkyl are optionally substituted by one to three moieties independently selected from the group consisting of hydroxy, halo, CN—, (C₁-C₆)alkyl, HO(C₁-C₆)alkyl, (C₁-C₆)alkyl-NH(C═O)—, NH₂(C═O)—, (C₁-C₆)alkoxy, or (C₃-C₈)cycloalkyl.

8. The method of claim 7 wherein R² is chloro, methyl or ethyl.

9. The method of claim 8 wherein R⁴ is hydrogen and R⁷ is —CH₂—C(R¹⁰R¹¹)—OH, wherein R¹⁰ and R¹¹ are independently selected from the group consisting of: hydrogen and (C₁-C₆)alkyl optionally substituted with (C₁-C₆)alkoxy- or —OH.

10. The method of claim 9 wherein R⁷ is hydrogen and R⁷ is selected from the group consisting of:

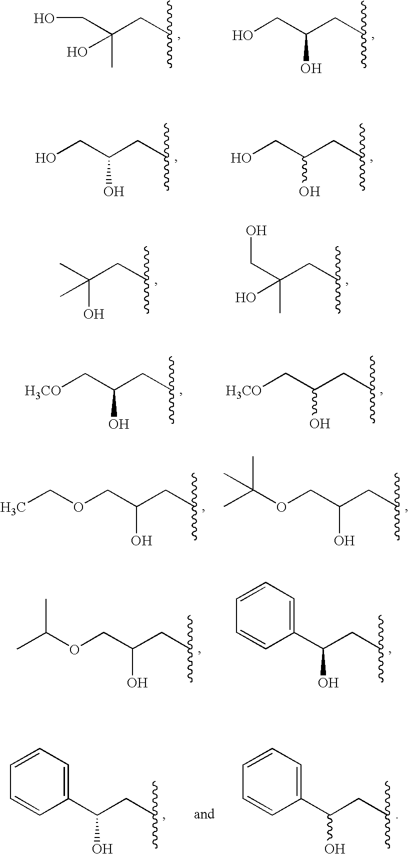

11. A method of preparing 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide, wherein said method comprises reacting 2-Chloro-5-(3,5-dioxo-4,5-dihydro-3H-[1,2,4]triazin-2-yl)-N-(1-hydroxy-cycloheptylmethyl)-benzamide with (R)-(−)-glycidyl methyl ether in the presence of at least one Lewis acid.

12. A composition of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising:
crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide; and
less than 2.5% residual organic solvent.

13. A process for preparing a composition of crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising less than 2.5% residual organic solvent comprising:
(a) combining n-heptane with a solution of acetone comprising 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide to generate crystals of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide; and
(b) isolating crystals of 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising less than 2.5% (w/w) residual organic solvent.

14. A method of treating a subject suffering from a disease selected from the group consisting of rheumatoid arthritis, ankylosing spondylitis, osteoarthritis, psoriatic arthritis, psoriasis, inflammatory diseases, and autoimmune diseases, the method comprising:
administering a therapeutically effective amount of crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising less than 2.5% residual organic solvent.

15. A pharmaceutical composition comprising:
a therapeutically effective amount of crystalline 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide comprising less than 2.5% residual organic solvent admixed with at least one pharmaceutically acceptable carrier.
